(19) United States
(12) Patent Application Publication
(10) Pub. No.: US 2009/0099376 A1
(43) Pub. Date: Apr. 16, 2009

(54) TIGECYCLINE AND METHODS OF PREPARING INTERMEDIATES

(75) Inventors: Michel Bernatchez, Montreal (CA); Warren Chew, Pierrefouds (CA); Sylvain Daigneault, Lavel (CA); Ernest Palus, Montreal (CA); Mahmoud Mirmehrabi, Lavel (CA); Luc Bouchard, La Prairie (CA)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/251,488

(22) Filed: Oct. 15, 2008

Related U.S. Application Data
(60) Provisional application No. 60/999,322, filed on Oct. 16, 2007.

(51) Int. Cl. C07C 237/26

(52) U.S. Cl. 552/205

(57) ABSTRACT

Methods of preparing and purifying 9-nitrominocycline and 9-aminominocycline and salts thereof used in the process of making tigecycline, are disclosed. In one embodiment, the invention is directed to a method of preparing the compound of formula 1

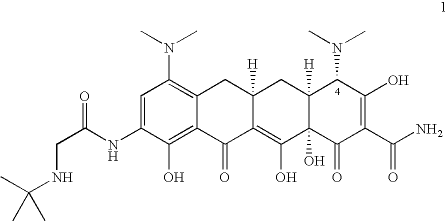

or a pharmaceutically acceptable salt thereof, comprising:
(a) reacting nitric acid with the compound of formula 2,

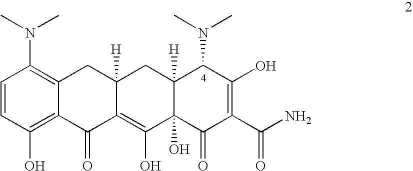

or a salt thereof, to produce a reaction mixture comprising an intermediate; and
(b) further reacting the intermediate to form the compound of formula 1, wherein the intermediate is isolated from the reaction mixture, the method further comprising sparging with an inert gas prior to step (a).

### CROSS-REFERENCE TO RELATED APPLICATIONS

This application claims the benefit under 35 U.S.C. §119(e) to co-pending U.S. Provisional Application Ser. No. 60/999,322, filed Oct. 16, 2007, which is hereby incorporated by reference in its entirety.

### FIELD OF INVENTION

The present invention relates to methods of preparing intermediates useful in the synthesis of tigecycline or a pharmaceutically acceptable salt thereof.

### BACKGROUND OF THE INVENTION

Tigecycline was developed in response to the worldwide threat of emerging resistance to antibiotics. Tigecycline has expanded broad-spectrum antibacterial activity both in vitro and in vivo. Glycylcycline antibiotics, like tetracycline antibiotics, act by inhibiting protein translation in bacteria.

Tigecycline, is known as GAR-936 and by the chemical name 9-(t-butylglycylamido)-minocycline, TBA-MINO), (4S,4aS,5aR,12aS)-9-[2-(tert-butylamino)acetamido]-4,7-bis(dimethylamino)-1,4,4a,5,5a,6,11,12a-octahydro-3,10,12,12a-tetrahydroxy-1,11-dioxo-2-naphthacenecarboxamide. Tigecycline is a glycylcycline antibiotic and an analog of the semisynthetic tetracycline, minocycline. Tigecycline is a 9-t-butylglycylamido derivative of minocycline.

Tigecycline is a known antibiotic in the tetracycline family and a chemical analog of minocycline. It may be used as a treatment against drug-resistant bacteria, and it has been shown to work where other antibiotics have failed. For example, it is active against methicillin-resistant Staphylococcus aureus, penicillin-resistant Streptococcus pneumoniae, vancomycin-resistant enterococci (D. J. Beidenbach et. al., Diagnostic Microbiology and Infectious Disease 40:173-177 (2001); H. W. Boucher et. al., Antimicrobial Agents & Chemotherapy 44:2225-2229 (2000); P. A. Bradford Clin. Microbiol. Newslett. 26:163-168 (2004); D. Milatovic et. al., Antimicrob. Agents Chemother. 47:400-404 (2003); R. Patel et. al., Diagnostic Microbiology and Infectious Disease 38:177-179 (2000); P. J. Petersen et. al., Antimicrob. Agents Chemother. 46:2595-2601 (2002); and P. J. Petersen et. al., Antimicrob. Agents Chemother. 43:738-744(1999), and against organisms carrying either of the two major forms of tetracycline resistance: efflux and ribosomal protection (C. Betriu et. al., Antimicrob. Agents Chemother. 48:323-325 (2004); T. Hirata et. al. Antimicrob. Agents Chemother. 48:2179-2184 (2004); and P. J. Petersen et. al., Antimicrob. Agents Chemother. 43:738-744(1999).

Tigecycline may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, tigecycline may be used to treat or control bacterial infections in warm-blooded animals caused by bacteria having the TetM and TetK resistant determinants. Also, tigecycline may be used to treat bone and joint infections, catheter-related Neutropenia, obstetrics and gynecological infections, or to treat other resistant pathogens, such as VRE, ESBL, enterics, rapid growing mycobacteria, and the like.

Tigecycline suffers some disadvantages in that it may degrade by epimerization. Epimerization is a known degradation pathway in tetracyclines generally, although the rate of degradation may vary depending upon the tetracycline. Comparatively, the epimerization rate of tigecycline may be fast, even for example, under mildly acidic conditions and/or at mildly elevated temperatures. The tetracycline literature reports several methods scientists have used to try and minimize epimer formation in tetracyclines. In some methods, the formation of calcium, magnesium, zinc or aluminum metal salts with tetracyclines limit epimer formation when done at basic pHs in non-aqueous solutions. (Gordon, P. N, Stephens Jr, C. R., Noseworthy, M. M., Teare, F. W., U.K. Patent No. 901,107). In other methods, (Tobkes, U.S. Pat. No. 4,038,315) the formation of a metal complex is performed at acidic pH and a stable solid form of the drug is subsequently prepared.

Tigecycline differs structurally from its epimer in only one respect. Wherein in tigecycline, the N-dimethyl group at the 4 carbon is cis to the adjacent hydrogen as shown below in formula I, whereas in the epimer (i.e., the C₄-epimer), formula II, they are trans to one another in the manner as indicated below. Although the tigecycline epimer is believed to be non-toxic, under certain conditions it may lack the anti-bacterial efficacy of tigecycline and may, therefore, be an undesirable degradation product. Moreover, the amount of epimerization can be magnified when synthesizing tigecycline in a large scale.

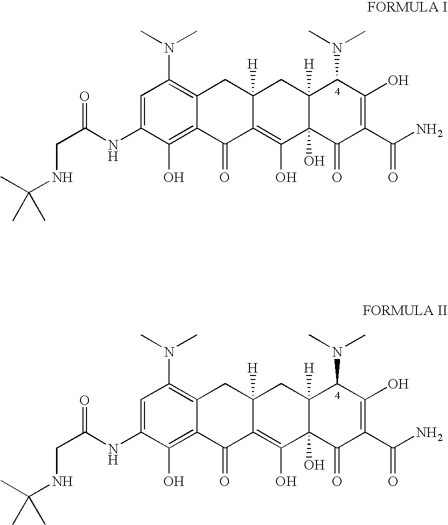

Other methods for reducing epimer formation include maintaining pHs of greater than about 6.0 during processing; avoiding contact with conjugates of weak acids such as formates, acetates, phosphates, or boronates; and avoiding contact with moisture including water-based solutions. With regard to moisture protection, Noseworthy and Spiegel (U.S. Pat. No. 3,026,248) and Nash and Haeger, (U.S. Pat. No. 3,219,529) have proposed formulating tetracycline analogs in non-aqueous vehicles to improve drug stability. However, most of the vehicles included in these disclosures are more appropriate for topical than parenteral use. Tetracycline epimerization is also known to be temperature dependent so production and storage of tetracyclines at low temperatures can also reduce the rate of epimer formation (Yuen, P. H., Sokoloski, T. D., J. Pharm. Sci. 66: 1648-1650,1977; Pawelczyk, E., Matlak, B, Pol. J. Pharmacol. Pharm. 34: 409-421, 1982). Several of these methods have been attempted with tigecycline but apparently none have succeeded in reducing both epimer formation and oxidative degradation while not introducing additional degradants. Metal complexation, for example, was found to have little affect on either epimer formation or degradation generally at basic pH.

Although the use of phosphate, acetate, and citrate buffers improve solution state stability, they seem to accelerate degradation of tigecycline in the lyophilized state. Even without a buffer, however, epimerization is a more serious problem with tigecycline than with other tetracyclines such as minocycline.

In addition to the C₄-epimer, other impurities include oxidation by-products which occur during the various steps of synthetic methods used to make tigecycline. Some of these by-products are obtained by oxidation of the D ring of the molecule, which is an aminophenol or oxidation at the C-11 and C-12a positions.

Moreover, degradation products may be obtained during each of the different synthetic steps of a synthetic scheme, and separating the required compound from these degradation products can be tedious. For example, conventional purification techniques, such as chromatography on silica gel or preparative HPLC cannot be used to purify these compounds easily because of their chelating properties. Although some tetracyclines have been purified by partition chromatography using columns made of diatomaceous earth impregnated with buffered stationary phases containing sequestering agents like EDTA, these techniques can suffer from very low resolution, reproducibility and capacity. These disadvantages may hamper a large-scale synthesis. HPLC has also been used for purification, but adequate resolution of the various components on the HPLC columns requires the presence of ion-pairing agents in the mobile phase. Separating the final product from the sequestering and ion-pairing agents in the mobile phase can be difficult.

While on a small-scale the impure compounds obtained by precipitation may be purified by preparative reverse-phase HPLC, purification by reverse phase liquid chromatography can be inefficient and expensive when dealing with kilogram quantities of material.

Accordingly, there remains a need to obtain intermediates and tigecycline in a more purified form than previously achieved. There also remains a need for new processes to minimize the use of chromatography for purification of any or each of the individual large scale process steps.

### BRIEF SUMMARY OF THE INVENTION

Methods for producing tigecycline of formula I or a pharmaceutically acceptable salt thereof, are disclosed.

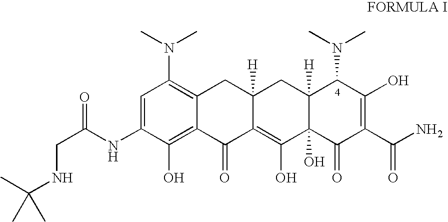

Also disclosed herein are methods for producing tigecycline, as illustrated in Scheme I.

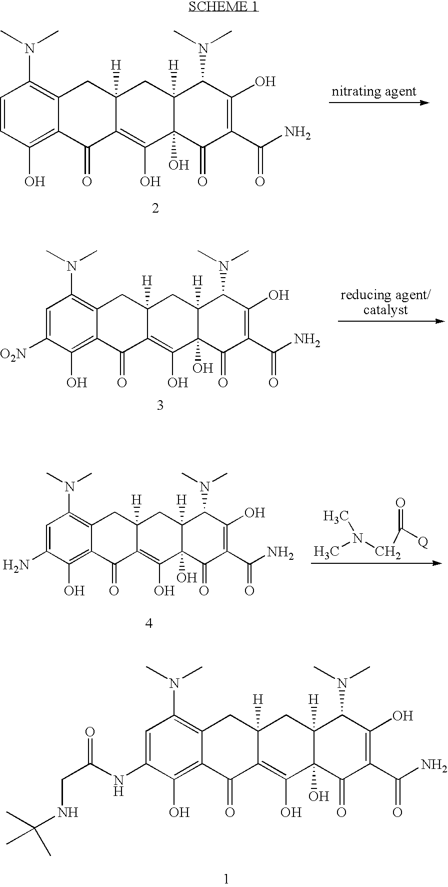

The compound of formula 2 is also known as a minocycline or minocycline derivative. Minocycline 2 is available commercially as the hydrochloride or sulfate salt. Reaction of minocycline of formula 2 with at least one nitrating agent results in a —NO₂ substituent to form the compound of formula 3. The —NO₂ substituent in formula 3 can be subsequently reduced to an amino, such as by hydrogenation, to form the compound of formula 4 as the sulfuric acid salt which is optionally converted to the HCL salt. Finally, acylation of the compound of formula 4 generates the compound of formula 1, tigecycline.

Disclosed herein are methods for performing reactions to produce tigecycline of formula 1, e.g., nitration, reduction, and acylation reactions and in particular the nitration and reduction reactions. Also disclosed are methods for purifying said nitration and reduction reaction products which are useful to produce tigecycline of formula 1 Other processes are discussed in: application Ser. No. 11/440,031, publication number 2007-0049560A1; application Ser. No. 11/440,038, publication number 2007-0049563A1; application Ser. No. 11/440,035, publication number 2007-0049562A1; and application Ser. No. 11/440,034, publication number 2007-0049561A1 which are herein incorporated by reference in their entirety.

The methods disclosed herein can form the desired product tigecycline while reducing the amount of at least one impurity present in the intermediate products, such as epimer formation, the presence of starting reagents, and oxidation by-products. Such reduction in impurities can be achieved through the intermediate steps and during at least one stage of the synthesis, especially during any one of the nitration or reduction reactions. The methods disclosed herein can also facilitate large scale synthesis with suitable purities of the final product tigecycline.

In particular, it has been found that the nitration step is beneficially performed to completion using nitric acid in the presence of sulfuric acid with minocycline hydrochloride using adequate stirring and while using vacuum to remove the presence of hydrogen chloride, to give formula 3. Further, it has also been found that the reduction of formula 3 with catalyst is beneficially performed to completion in a solvent mixture of water:methanol to give formula 4.

### DEFINITIONS

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

“Tigecycline” as used herein includes tigecycline in free base form and salt forms, such as any pharmaceutically acceptable salt, enantiomers, and epimers. Tigecycline, as used herein, may be formulated according to methods known in the art.

“Compound” as used herein refers to a neutral compound (e.g. a free base), and salt forms thereof (such as pharmaceutically acceptable salts). The compound can exist in anhydrous form, or as a hydrate, or as a solvate. The compound may be present as stereoisomers (e.g., enantiomers and diastereomers), and can be isolated as enantiomers, racemic mixtures, diastereomers, and mixtures thereof. The compound in solid form can exist in various crystalline and amorphous forms.

“Pharmaceutically acceptable” as used herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

“Adequate stirring is agitation of a reaction mixture at a sufficient speed (measured as revolutions per minute or rpm) to achieve desired reaction outcome. The ‘rpm’ range is dependent on the size of the reaction vessel, volume of reaction mixture, the diameter and type of agitating impeller. It varies between ˜50 rpm to ˜500 rpm depending on scale of reaction.

Each of the various embodiments of the invention will be described as follows.

### Nitration

One embodiment discloses a nitration reaction where the product of the nitration is isolated. Accordingly, in one embodiment, the method comprises:

(a) reacting nitrating agent, nitric acid, with minocycline of formula 2,

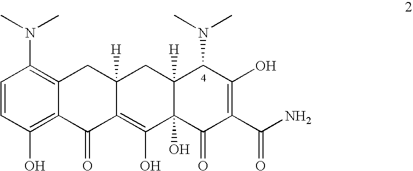

or a salt thereof, to produce a reaction mixture comprising an intermediate; and

further reacting the intermediate to form tigecycline of formula 1

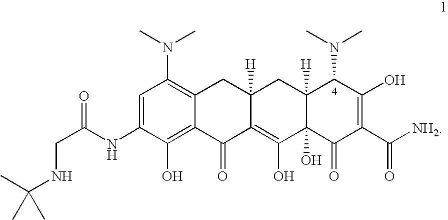

The minocycline of formula 2 can be provided as a free base or as a salt. In one embodiment, the minocycline of formula 2 is a hydrochloride salt. “Salts” as used herein may be prepared in situ or separately by reacting a free base with a suitable acid. Exemplary salts include, but are not limited to, hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts. In another embodiment, the salt can be chosen from alkylsulfonic and arylsulfonic salts. In one embodiment, minocycline of formula 2 is provided as a hydrochloride salt, or as a sulfate salt.

“Nitrating agent” as used herein refers to a reagent that can add a —NO₂ substituent to a compound, or transform an existing substituent to an —NO₂ substituent. Exemplary nitrating reagents include nitric acid and nitrate salts, such as alkali metal salts, e.g., KNO₃. Wherein the nitrating agent is nitric acid, the nitric acid can have a concentration of at least 90%, such as a concentration of 90%, 95%, 99%, or even 100%. In one embodiment the nitrating agent is nitric acid of at least or greater than 90%.

The nitrating agent, nitric acid can react with minocycline of formula 2 in any solvent deemed suitable by one of ordinary skill in the art. In one embodiment, the reaction is performed in the presence of sulfuric acid and/or sulfate salts. In one embodiment, the sulfuric acid used is concentrated sulfuric acid, e.g., a concentration of at least 50%, 60%, 70%, 80%, 85%, 90%, or at least 95%.

In one embodiment, nitrating agent nitric acid is provided in a molar excess relative to the compound of formula 2. Suitable molar excesses can include, but are not limited to, values such as at least 1.05, e.g., a molar excess ranging from 1.05 to 1.75 equivalents, such as a molar excess ranging from 1.05 to 1.5, or from 1.05 to 1.25, or from 1.05 to 1.1 equivalents. In another embodiment, the molar excess is 1.05, 1.1, 1.2, 1.3, or 1.4 equivalents. In a further embodiment, the molar excess is 1.2 to 1.5 equivalents.

In one embodiment, the nitration reaction is performed under vacuum. In one embodiment the vacuum is 50 to 300 torr. In a further embodiment the vacuum is 20 to 50 torr. In a further embodiment the vacuum is less than 20 torr.

In one embodiment, nitrating agent, nitric acid is reacted with minocycline of formula 2 by adding the nitric acid over a period of time. In one embodiment the minocycline of formula 2 is the hydrochloride salt. One of ordinary skill in the art can determine a time period over which the total amount of nitrating agent, nitric acid is added to optimize the reaction conditions using analytical methods which include HPLC. The addition of nitration reagent, nitric acid can be monitored by, for example by HPLC, to control the amount of nitrating agent used. In one embodiment, the total amount of nitrating agent is added over a period of time of at least 1 h, such as a period of time of at least 2 h, at least 3 h, at least 5 h, at least 10 h, at least 24 h, or a period of time ranging from 1 h to 1 week, ranging from 1 h to 48 h, ranging from 1 h to 24 h, or ranging from 1 h to 12 h. In a further embodiment following the reaction with the nitrating agent there is a period of time before isolation of the desired product.

In an embodiment, the nitric acid can be added continuously.

In one embodiment, the nitric acid is added under inert gas.

In one embodiment, nitric acid can be reacted with minocycline of formula 2 at a temperature ranging from 0 to 25° C., such as a temperature ranging from 0 to 15° C., from 5 to 10° C., or from 10 to 15° C. In one embodiment the temperature range is 3 to 7° C. An “intermediate” as used herein refers to a compound that is formed as an intermediate product between the starting material and the final product. In one embodiment, the intermediate of formula 3 or a salt thereof is a product of the nitration of minocycline of formula 2 with a nitration agent, nitric acid under vacuum with adequate stirring.

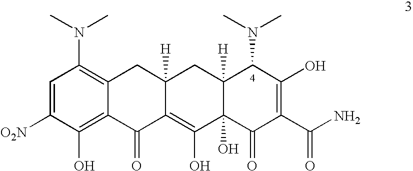

The intermediate can exist as a free base or as a salt, such as any of the salts disclosed herein. In one embodiment, the intermediate is a sulfate salt.

In one embodiment, the intermediate of formula 3 is not isolated from the reaction mixture. “Reaction mixture” as used herein refers to a solution or slurry comprising at least one product of a chemical reaction between reagents, as well as by-products, e.g., impurities (including compounds with undesired stereochemistries), solvents, and any remaining reagents, such as starting materials. In one embodiment, the intermediate of formula 3 is the product of the nitration and is present in the reaction mixture, which can also contain starting reagents (such as the nitrating agent and/or minocycline of formula 2), by-products (such as the C₄-epimer of either formula 2 or formula 3). In one embodiment, the reaction mixture is a slurry, where a slurry can be a composition comprising at least one solid and at least one liquid (such as water, acid, or a solvent), e.g., a suspension or a dispersion of solids. In a further embodiment the reaction mixture is a solution. In one embodiment the intermediate of formula 4 is isolated substantially free of minocycline. By substantially free applicants mean that minocycline is present in less than 5.0% to about 0.1%.

In one embodiment, the nitration reaction produces the intermediate while generating a low amount of the corresponding C₄-epimer. For example, where the intermediate of formula 3, the nitration results in the formation of C₄-epimer of formula 3 in an amount less than 5%, less than 3%, less than 2%, less than 1%, or 1.42-1.96% as determined by high performance liquid chromatography (HPLC).

HPLC parameters for each step, i.e., nitration, and reduction, are provided in the Examples section.

In one embodiment, the nitration is performed such that the amount of starting material, e.g., the minocycline of formula 2, remaining in the reaction mixture is 5% to non-detected (less than 0.1%). In one embodiment minocycline of formula 2 is present at non-detectable levels (less than <0.1%).

In one embodiment, the nitration can be performed in a large scale. In one embodiment, “large scale” refers to the use of at least 1 gram of minocycline of formula 2, such as the use of at least 2 grams, at least 5 grams, at least 10 grams, at least 25 gram, at least 50 grams, at least 100 grams, at least 500 g, at least 1 kg, at least 5 kg, at least 10 kg, at least 25 kg, at least 50 kg, at least 100 kg or at least 200 kg.

In one embodiment, the reduced form is a compound of formula 4,

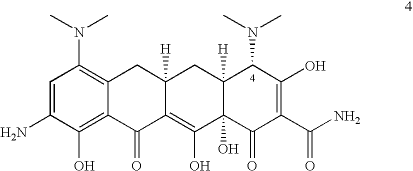

or a salt thereof. In one embodiment the salt is the sulfuric acid salt and in one embodiment the salt in the HCl salt. In one embodiment the sulfuric acid salt is converted to the HCl salt.

In one embodiment, the further reacting comprises reducing the intermediate 3. In another embodiment, the method further comprises acylating the reduced intermediate 4 to provide tigecycline of formula 1.

Another embodiment disclosed herein is a method of preparing a compound tigecycline of formula 1,

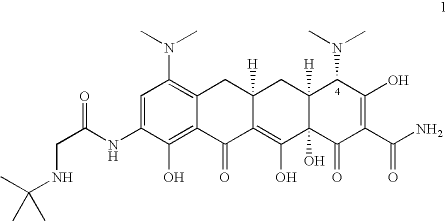

or a pharmaceutically acceptable salt thereof,

comprising:

(a) reacting at least one nitrating agent, nitric acid with minocycline of formula 2,

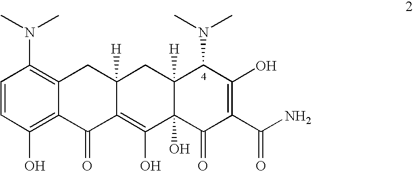

or a salt thereof, to produce a reaction mixture comprising an intermediate 3; and

(b) further reacting the intermediate 3 to form tigecycline of formula 1,

In one embodiment, the intermediate 3 is isolated from the reaction mixture.

In one embodiment, the compound of formula 1 is tigecycline.

Another embodiment disclosed herein is a method of preparing the compound of formula 1, tigecycline,

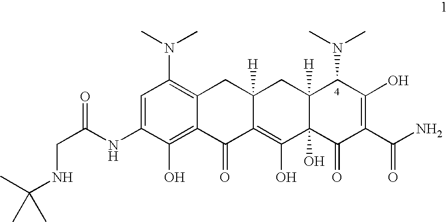

or a pharmaceutically acceptable salt thereof,

comprising:

(a) reacting at least one nitrating agent, nitric acid with minocycline of formula 2,

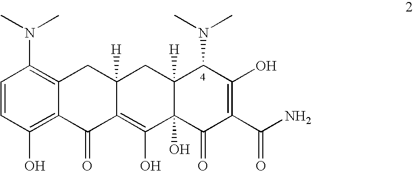

or a salt thereof, to produce a solution; and

(b) further reacting the solution to form tigecycline of formula 1.

Another embodiment disclosed herein is a method of preparing the compound of formula 3 or a salt thereof,

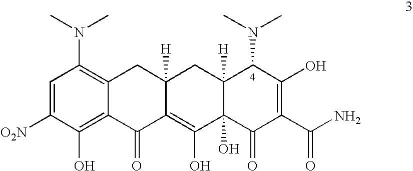

comprising:

reacting at least one nitrating agent, nitric acid, with minocycline hydrochloride of formula 2 or a salt thereof,

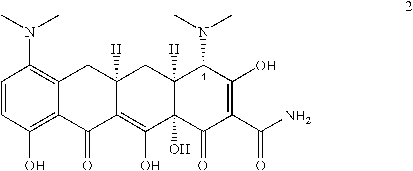

wherein the reacting is performed at a temperature ranging from 0 to 7° C.

Another embodiment disclosed herein is a method of preparing tigecycline of formula 1,

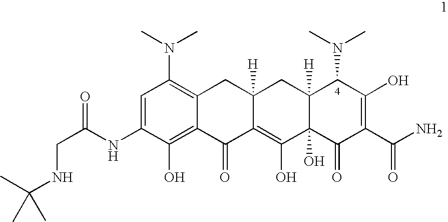

or a pharmaceutically acceptable salt thereof,

comprising:

reacting at least one nitrating agent, nitric acid, with minocycline of formula 2 or a salt thereof

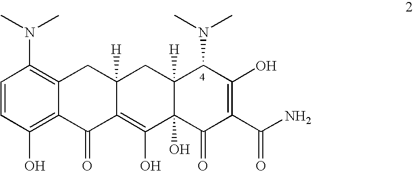

at a temperature ranging from 0 to 15° C.

to produce a reaction mixture comprising an intermediate of formula 3;

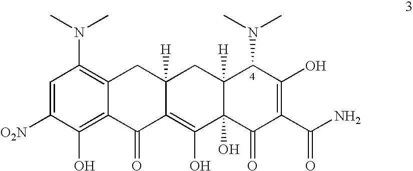

further reacting the intermediate of formula 3 to form the at least one compound of formula 1.

In one embodiment the temperature range is 5 to 10° C.

In one embodiment the temperature range is 0 to 15° C.

In one embodiment the temperature range is 3 to 7° C.

### Reduction

One embodiment discloses a method of preparing a compound of formula 4,

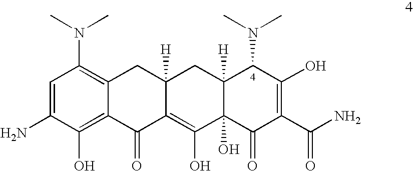

or a salt thereof,

comprising:

combining at least one reducing agent with a reaction mixture, such as a reaction mixture slurry or solution comprising an intermediate prepared from a reaction between at least one nitrating agent, nitric acid and minocycline hydrochloride of formula 2,

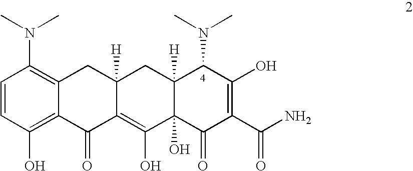

or a salt thereof.

In one embodiment, the method describes a process, where the nitration and reduction steps are independently performed with isolating the products of the nitration from the nitration reaction mixture. In one embodiment the products of the nitration, formula 3 and the reduction formula 4 are independently isolated.

“Reducing agent” as used herein refers to a chemical agent that adds hydrogen to a compound. In one embodiment, a reducing agent is hydrogen. The reduction can be performed under a hydrogen atmosphere at a suitable pressure as determined by one of ordinary skill in the art. In one embodiment, the hydrogen is provided at a pressure ranging from 1 to 75 psi, such as a pressure ranging from 60 to 70 psi, 1 to 50 psi, or a pressure ranging from 1 to 40 psi or a pressure of 70 psi.

In another embodiment, the reducing agent is provided in the presence of at least one catalyst. Exemplary catalysts include, but are not limited to, rare earth metal oxides, Group VIII metal-containing catalysts, and salts of Group VIII metal-containing catalyst. An example of a Group VIII metal-containing catalyst is palladium, such as palladium-on-carbon. In an embodiment palladium is used as 5-10% palladium on carbon (50% water wet). In one embodiment where the catalyst is palladium on carbon, the catalyst is present in an amount ranging from 2.5 wt % to 5.0 wt %, relative to the amount of 9-nitrominocycline of formula 3 present prior to the reaction.

One of ordinary skill in the art can determine a suitable solvent for the reduction reaction. In one embodiment, prior to the combining, e.g., prior to the reduction, the reaction mixture is combined with a solvent comprising at least one (C₁-C₈) alcohol. The at least one (C₁-C₈) alcohol can be chosen, for example, from methanol and ethanol. In one embodiment the (C₁-C₈) alcohol is methanol with water as a cosolvent. In one embodiment the methanol is in the range of 20 to 99%. In one embodiment the water is in the range of 1% to 80%. In another embodiment the ratio of water to methanol is 80:20. In another embodiment the reduction reaction is optionally performed in the presence of sulfuric acid.

One of ordinary skill in the art can determine a suitable temperature for the reduction reaction. In one embodiment, the combining, e.g., the reduction, is performed at a temperature ranging from 0° C. to 50° C., such as a temperature ranging from 0° C. to 5° C., 0° C. to 10° C., 20° C. to 40° C., or a temperature ranging from 26° C. to 28° C.

In one embodiment, after the combining, e.g., after the reduction, the resulting reaction mixture is added to or combined with a solvent system comprising a (C₁-C₈) branched chain alcohol and a (C₁-C₈) hydrocarbon. In one embodiment, the (C₁-C₈) branched chain alcohol is isopropanol. In one embodiment, the (C₁-C₈) hydrocarbon is chosen from hexane, heptane, and octane.

In one embodiment, after the combining, e.g., after the reduction, the resulting reaction mixture is added to the solvent system at a temperature ranging from 0° C. to 50° C., such as a temperature ranging from 0° C. to 10° C.

In one embodiment, the method further comprises isolating the at least one compound of formula 4 as a solid, or as a solid composition. In one embodiment, the at least one compound of formula 4 is precipitated or isolated as a salt, such as any of the salts described herein. In one embodiment the compound of formula 4 is isolated as the sulfuric acid salt. In one embodiment the compound of formula 4 is isolated as the HCl salt. In one embodiment the sulfuric acid salt is converted to the HCl salt.

In one embodiment, the solid composition comprises a C₄-epimer of formula 4 in an amount less than less than 5%, less than 3%, less than 2%, less than 1%, or less than 0.5% as determined by high performance liquid chromatography.

In one embodiment, the solid composition comprises the compound of formula 2 in an amount less than 2%, such as an amount less than 1%, or less than 0.5%, as determined by high performance liquid chromatography.

In one embodiment, the reduction can be performed in a large scale. In one embodiment, “large scale” refers to the use of at least 1 gram of the compound according to formula 2, such as the use of at least 2 grams, at least 5 grams, at least 10 grams, at least 25 gram, at least 50 grams, at least 100 grams, at least 500 g, at least 1 kg, at least 5 kg, at least 10 kg, at least 25 kg, at least 50 kg, or at least 100 kg.

Another embodiment disclosed herein is a method of preparing tigecycline of formula 1,

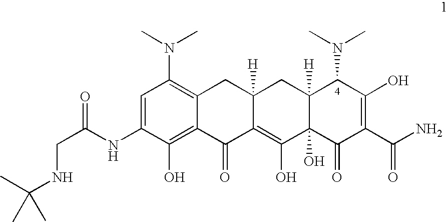

or a pharmaceutically acceptable salt thereof,

comprising:

(a) combining at least one reducing agent with a reaction mixture, such as a reaction mixture slurry, comprising an intermediate prepared from a reaction between at least one nitrating agent, nitric acid and minocycline of formula 2,

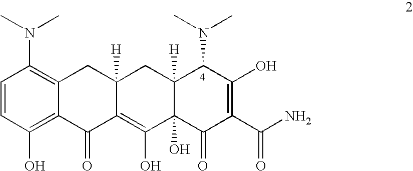

or a salt thereof, to form a second intermediate; and

(b) further reacting the second intermediate in the reaction mixture to prepare the compound of formula 1, tigecycline.

In one embodiment, the second intermediate is formula 4,

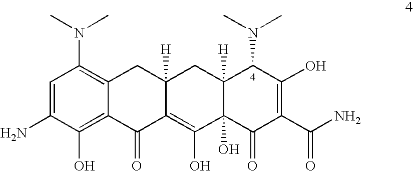

or a salt thereof.

In one embodiment, further reacting intermediate 4 comprises acylating the second intermediate. In one embodiment, prior to the acylating, the second intermediate can be precipitated or isolated as a salt. In one embodiment the salt is the sulfuric acid salt. In one embodiment the salt is the HCl salt.

Another embodiment disclosed herein is a method of preparing a compound of formula 4 or a salt thereof,

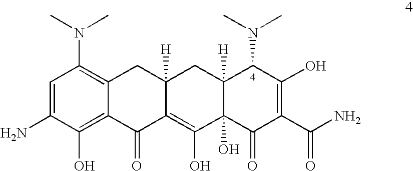

comprising:

reducing an intermediate of formula 3 or a salt thereof,

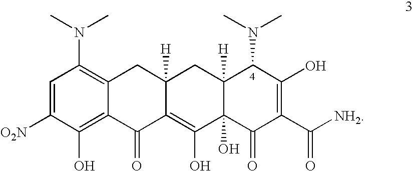

In one embodiment, the intermediate of formula 3 may be present in a reaction mixture solution.

In one embodiment, the reducing comprises combining at least one reducing agent with the reaction mixture.

Another embodiment disclosed herein is a method of preparing tigecycline of formula 1,

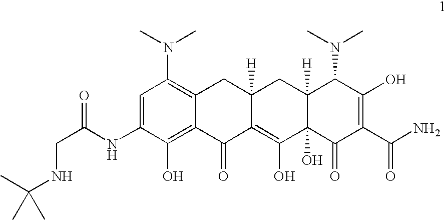

or a pharmaceutically acceptable salt thereof,

comprising:

(a) reacting at least one nitrating agent, nitric acid, with minocycline of formula 2 or a salt thereof to prepare a reaction mixture,

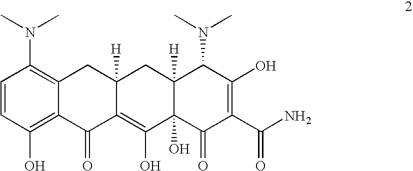

(b) isolating intermediate 3 and combining at least one reducing agent with the reaction mixture to prepare an intermediate; and

(c) preparing tigecycline of formula 1 from the intermediate.

Another embodiment disclosed herein is method of preparing tigecycline of formula 1,

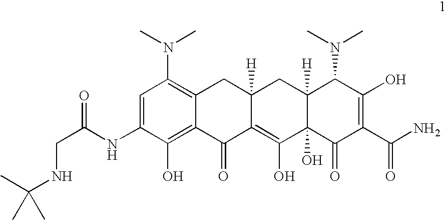

or a pharmaceutically acceptable salt thereof,

comprising:

(a) combining at least one Group VIII metal-containing catalyst in the presence of hydrogen with a reaction mixture, such as a reaction mixture slurry, prepared from a reaction between a nitrating agent, nitric acid and minocycline of formula 2 or a salt thereof,

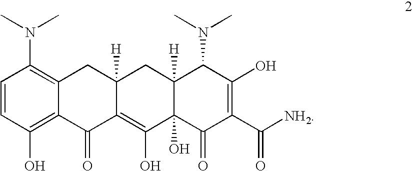

In one embodiment, the at least one Group VIII metal-containing catalyst is present in an amount ranging from 0.1 parts to 1 part relative to the amount of formula 2 present prior to the reaction with the at least one nitrating agent.

Another embodiment disclosed herein is a composition comprising:

a compound of formula 4,

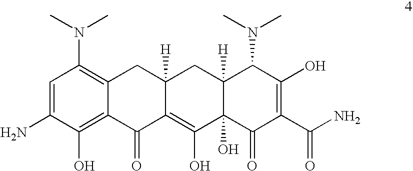

or a salt thereof,

wherein a C₄-epimer of formula 4 is present in an amount ranging from 1.49% to 2.95% as determined by high performance liquid chromatography.

One embodiment of the disclosure includes a method for preparing at the compound of Formula 1:

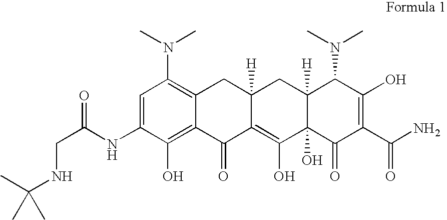

or a pharmaceutically acceptable salt thereof,

comprising:

A) reacting at least one nitrating agent, nitric acid, with the compound of Formula 2:

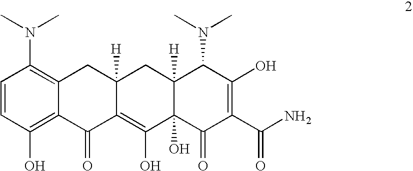

or a salt thereof,

to prepare a reaction mixture comprising at least one compound of Formula 3:

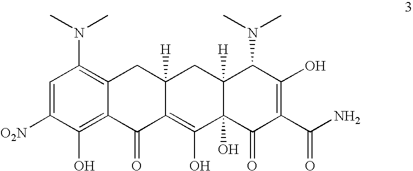

or a salt thereof,

B) combining at least one reducing agent with the reaction mixture slurry to prepare at least one compound of Formula 4,

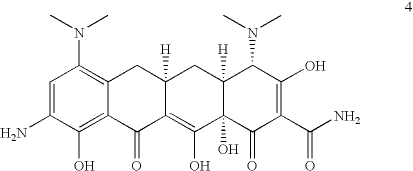

or a salt thereof, and

C) reacting the compound of Formula 4 with the aminoacyl compound 6 in a reaction medium chosen from an aqueous medium, and at least one basic solvent in the absence of a reagent base.

The compound formula I prepared by this method is tigecyline.

In one embodiment, Formula 1 is [4S-(4α,12aα)]-4,7-Bis(dimethylamino)-9-[[(t-butylamino)acetyl]amino]-1,4,4a,5,5a,6,11,12a-octahydro-3,10,12,12a-tetrahydroxy-1,11-dioxo-2-naphthacene-carboxamide, such as pharmaceutically acceptable salts such as HCl salts.

One embodiment of the disclosure includes a method for preparing the compound of Formula 1:

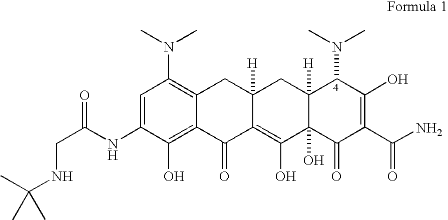

or a pharmaceutically acceptable salt thereof,

comprising:

a) reacting at least one nitrating agent, nitric acid with at least one compound of Formula 2:

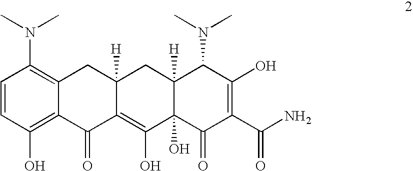

or a salt thereof,

to prepare a reaction mixture, such as a reaction mixture slurry, comprising an intermediate of Formula 3:

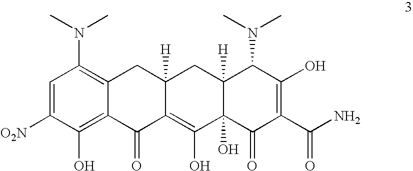

or a salt thereof,

b) combining at least one reducing agent with the reaction mixture slurry to prepare a second intermediate of Formula 4,

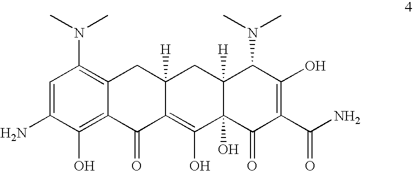

or a salt thereof,

c) reacting the second intermediate with at least one aminoacyl compound 6 in a reaction medium to obtain the compound of formula 1. In one embodiment, the reaction medium is chosen from an aqueous medium, and at least one basic solvent in the absence of a reagent base. Additional steps may include, for example at least one of:

d) combining the compound of Formula 1 with at least one polar aprotic solvent and at least one polar protic solvent to give a first mixture,

e) mixing the first mixture for at least one period of time, such as ranging from 15 minutes to 2 hours, at a temperature, such as ranging from 0° C. to 40° C., and

f) obtaining the compound of Formula 1. In one embodiment, any of the intermediates 3 or 4 of the methods disclosed may be isolated or precipitated out. In another embodiment, two or more steps of any of the methods disclosed are “one-pot” procedures.

Another embodiment of the disclosure includes a method for preparing the compound of Formula 1:

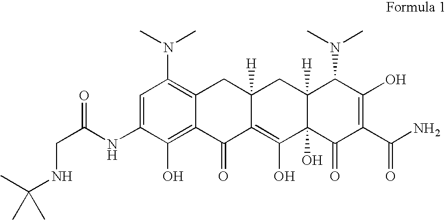

or a pharmaceutically acceptable salt thereof,

comprising:

a) combining at least one reducing agent with a reaction mixture, such as a reaction mixture slurry, comprising the compound of Formula 3:

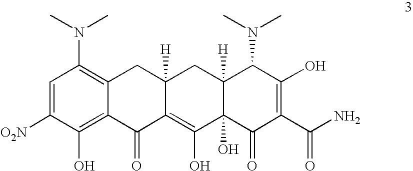

or a salt thereof, to prepare the compound of Formula 4,

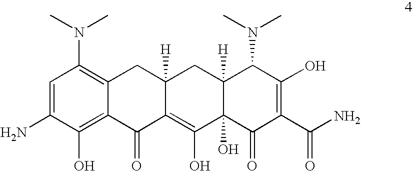

or a salt thereof,

b) reacting the intermediate 4 with the aminoacyl compound 6 in a reaction medium chosen from an aqueous medium to obtain the compound of Formula 1. In one embodiment, the reaction medium may be chosen from at least one basic solvent in the absence of a reagent base. Additional steps may include, for example, at least one of:

c) combining the compound of Formula 1 with at least one polar aprotic solvent and at least one polar protic solvent to give a first mixture,

d) mixing the first mixture for at least one period of time, such as ranging from 15 minutes to 2 hours, at a temperature, such as ranging from 0° C. to 40° C., and

e) obtaining the compound of Formula 1.

A further embodiment of the disclosure includes a method for preparing the compound of Formula 1:

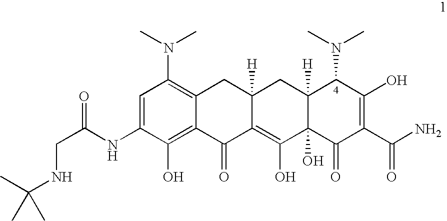

or a pharmaceutically acceptable salt thereof,

a) reacting the compound of Formula 4:

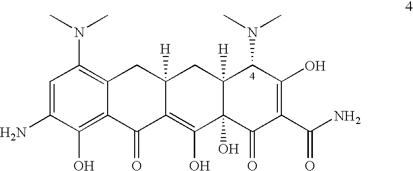

or a salt thereof,

with at least one aminoacyl compound in a reaction medium, for example, chosen from an aqueous medium, and at least one basic solvent in the absence of a reagent base to obtain the compound of Formula 1. Additional steps may include at least one of:

b) combining the compound of Formula 1 with at least one polar aprotic solvent and at least one polar protic solvent to give a first mixture,

c) mixing the first mixture for at least one period of time, such as ranging from 15 minutes to 2 hours, at a temperature, such as ranging from 0° C. to 40° C., and

d) obtaining the compound of Formula 1.

Any of these methods disclosed herein are for preparing a compound of Formula 1 may be a method for preparing a compound of Formula 1.

The terms “pharmaceutically acceptable salt” and refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Pharmaceutically acceptable salts may be derived from amino acids, including but not limited to cysteine. Other acceptable salts may be found, for example, in Stahl et al., Pharmaceutical Salts: Properties, Selection, and Use, Wiley-VCH; 1st edition (Jun. 15, 2002).

Other than in the examples, and where otherwise indicated, all numbers used in the specification and claims are to be understood as modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

### DETAILED DESCRIPTION OF THE INVENTION

The following described examples are intended to illustrate the invention in a nonlimiting manner.

### Tigecylcine Synthesis

The manufacturing process for tigecycline is a multi-step synthesis as shown in Scheme I. Briefly, minocycline hydrochloride dissolved in concentrated sulfuric acid was nitrated with concentrated nitric acid. The isolated 9-nitrominocycline sulfate salt intermediate dissolved in a mixture of methanol/water was hydrogenated with 5-10% palladium on carbon to provide 9-aminominocycline sulfate salt. The sulfate salt intermediate was subsequently converted to the 9-aminocycline hydrochloride salt in aqueous hydrochloric acid. The hydrochloride salt intermediate was treated with N-t-butylglycine acid chloride hydrochloride in water, purified from acetone/methanol and crystallized from methanol/dichloromethane to produce tigecycline.

### Nitration Reaction

Features in the nitration step of the chemical process included HCl purge, nitric acid addition time, nitric acid location of addition, mixing speed and nitric acid equivalents play a role in quality of compound of formula 3, 9-nitrominocycline produced. Directly related to these parameters are the levels of HCl, chloro-impurity (impurity A) and nitro ester (impurity B) levels of the impurity generation that could affect the quality of the product.

### Hydrogenation Reduction Step

Parameters in this step were evaluated for its potential risk for failure or incomplete reaction. Parameters were listed in four areas: compound of formula 3, 9-nitrominocycline starting material, chemical process, operation and equipment associated with hydrogenation.

Identified parameters are the solvent ratio in the chemical process, the chloro (impurity A) and nitro ester (impurity B) impurities, and IPA residual solvent and purity.

The identified parameters with the defined study range are listed below.
1. Solvent—80:20 water:MeOH and 99:1 MeOH:water
2. Impurity A—0 to 10%
3. Impurity B—0 to 25%
4. IPA—4 to 50% (w/w)

The Argonaut (Biotage) Endeavor hydrogenation unit, equipped with 8-parallel pressure reactors, was used for a preliminary parallel screen. The compound of formula 3, 9-nitrominocycline starting material used for the screen was a batch with a purity of 70.6% and containing <0.1% impurity A and 8.0% impurity B. Enriched impurity A (61%) and enriched impurity B (19%) were used for spiking experiments. To prepare the samples with up to 10% impurity A, 3.54 g of 9-nitrominocycline was mixed with 0.46 g of enriched impurity A. HPLC analysis of the mixture shows purity: 61.5%, impurity A 12.8%, impurity B 6.1%.

Table 1 shows the design layout of 8 experiments conducted on 320 mg scale with a maximum volume of 2 ml. Scaling down the hydrogenation, the estimated agitation rate based on the geometric similarity equation was calculated to be 785 rpm. The limitation on the Endeavour hydrogenation unit permitted us to perform the experiments at 500 rpm.

TABLE 1
2-LEVEL 3-FACTOR DOE OF THE HYDROGENATION OF
9-NITROMINOCYCLINE

| | Factor 1 | | | |
|---|---|---|---|---|
| | Impurity A | Factor 2 | Factor 3 | |
| Run | (%) | IPA (%) | Solvent | SM (%) |
| 1 | 0.00 | 0.00 | 99% MeOH | 51 |
| 2 | 0.00 | 0.00 | 80% H₂O | nd* |
| 3 | 0.00 | 50.00 | 99% MeOH | 61 |
| 4 | 0.00 | 50.00 | 80% H₂O | nd |
| 5 | 10.00 | 0.00 | 99% MeOH | 49 |
| 6 | 10.00 | 0.00 | 80% H₂O | nd |
| 7 | 10.00 | 50.00 | 99% MeOH | 59 |
| 8 | 10.00 | 50.00 | 80% H₂O | nd |

*Not detected.

### Nitration of Minocycline

### Nitric Acid Quantity and Concentration

The nitric acid used was at least 90% and recommended nitric acid concentration for this reaction was ≧90%. The equivalent amount of nitric acid recommended for nitration was in the 1.2 to 1.5 equivalents range. In some experiments the nitric acid concentrations used were 91.0% and 90.1%.

### Effect of Minocycline Salt on Quality of 9-nitrominocycline

The impurity profile of 9-nitrominocyline produced in the nitration reaction, using minocycline sulfate vs. minocycline HCl was examined and the purity data is shown in Table 2.

TABLE 2
PURITY OF 9-NITROMINOCYCLINE PRODUCED
FROM MINOCYCLINE

| Sample type | Purity (%) | Imp A (%) | Imp B (%) |
|---|---|---|---|
| Minocycline sulfate | 95.3 | — | — |
| 9-nitrominocycline from | 76.3 | <0.1 | 5.5 |
| minocycline sulfate | | | |
| 9-nitrominocycline from | 61.6 | 0.1 | 8.8 |
| minocycline | | | |
| hydrochloride | | | |

A complete purity profile data table showing all nitration reactions, inclusive of impurities, performed is included in Table 15 hereinbelow.

Reaction parameters (minocycline sulfate): 16.5 g starting material, 50 ml (3 vols) sulfuric acid, 1.18 eq nitric acid, 100 ml 3-neck round-bottom flask (˜6 cm diameter), ˜4 cm length impeller. Stirring rate not measured. Nitrogen flow rate not measured.

Reaction parameters (minocycline HCl): 50 g starting material, 150 ml (3 vols) sulfuric acid, 1.53 eq nitric acid, 250 ml multi-neck round bottom flask. Impeller size, stirring rate and nitrogen flow rate not measured. Chloride ion content not measured.

A better purity of 9-nitrominocycline can be obtained if nitration is performed on minocycline sulfate vs minocycline HCl. These results substantiate the observation that the presence of HCl has a detrimental effect on the quality of the 9-nitrominocycline produced. Residual HCl will react with nitric acid thus making the latter inaccessible for nitration as shown in Schemes A and B below.

### Effect of Residual HCl on 9-nitrominocycline Purity

A more controlled set of experiments to monitor the effect of residual HCl on the purity of 9-nitrominocycline was conducted. These experiments were performed on 50 g scale in a 1-L ChemGlass cylindrical reactor, impeller diameter 5 cm and mixing rate 500 rpm. Data in Table 3 summarizes the purity of 9-nitrominocycline obtained at various levels of residual HCl in the starting material mixture just before nitration began. Nitric acid was added over 100 mins from the middle of the reactor vessel.

Data shows the purity of 9-nitrominocycline diminished as residual HCl increased. As HCl is increased to >1000 ppm, the purity dramatically declines. Concurrently, the level of Impurity A also increased as residual HCl increased. These results confirm the detrimental effects of residual HCl has on the nitration reaction. Residual HCl produces high levels of impurity A. However, with the increase in the level of impurity A (up to 10%), little effect on the subsequent hydrogenation in 80:20 water:methanol solvent mixture was noted.

TABLE 3
EFFECT OF RESIDUAL HCL ON PURITY OF
9-NITROMINOCYCLINE

| | Purity 9- | | | |
|---|---|---|---|---|
| | nitro- | | Impurity B | Impurity |
| Residual | minocycline | | (%) (rrt | B (%) (rrt |
| HCl (ppm) | (%) | Impurity A (%) | 0.59) | 0.68) |
| 68 | 69.12 | 0.43 | 8.12 | 6.33 |
| 482 | 65.91 | 5.87 | 7.06 | 5.22 |
| 1332 | 47.96 | 23.93 | 6.70 | 5.62 |

Nitration conducted on minocycline sulfate reached completion with only 1.2eq nitric acid. In control reactions using minocycline hydrochloride, 1.5eq nitric acid was typically required. This can be explained by the low chloride content of minocycline sulfate (106 ppm). At low concentrations of chloride, nitric acid will be available for nitration and not react with HCl. Dissolved HCl consumes nitric acid to produce nitric oxide and chlorine gas (Scheme A) or nitrosyl chloride (Scheme B). In either path, chlorine gas is produced that can react with minocycline leading to Impurity A. This leads to the necessity to use excess nitric acid to complete the nitration. These results support the conclusion that residual HCl is a parameter in the nitration step.

### Scheme A. Reaction of HCl with Nitric Acid (Path 1)

2 HNO₃+6 HCl→2 NO+3 Cl₂+4 H₂O

### Scheme B. Reaction of HCl with Nitric Acid (Path 2)

HNO₃+3 HCl→NOCl+Cl₂+2 H₂O

### Effect of Sparging on Hydrogen Chloride Removal

Generally, nitration may take place by contacting minocycline or a salt thereof with a reaction medium within a reactor vessel, wherein the reaction medium forms a surface defining a headspace portion above the surface and a subsurface portion beneath the surface, the method comprising (a) sparging the headspace portion without sparging the subsurface portion, (b) sparging the subsurface portion without sparging the headspace portion, or (c) sparging the headspace portion and the subsurface portion.

Minocycline is preferably dissolved in the reaction medium. The method may comprise sparging the headspace portion without sparging the subsurface portion; sparging the subsurface portion without sparging the headspace portion; or sparging the headspace portion and the subsurface portion.

The salt of minocycline may be a hydrochloride, wherein sparging decreases the amount of hydrogen chloride in the reactor vessel. Preferably, the amount of hydrogen chloride is decreased by up to 95%.

A study was conducted on the effect of inert gas (such as nitrogen) sparging and use of vacuum on gaseous hydrogen chloride removal prior to addition of nitric acid. Reaction parameters set for each experiment were as follows: 40 g minocycline, multi-neck 250 ml round-bottom flask (diameter 8 cm), impeller length 4 cm, mixing speed 100 rpm (i.e. under poor mixing conditions). The following experiments were performed:
1. Nitrogen sparging in the headspace (˜50 ml/min);
2. Nitrogen sparging sub-surface (˜50 ml/min);
3. Vacuum (300 torr);
4. No sparging.
   Samples of dissolved minocycline in sulfuric acid at selected times were analyzed for chloride content as shown in Table 4. The chloride content of minocycline hydrochloride was 6.8%.

TABLE 4
CHLORIDE CONTENT OF MINOCYCLINE IN SULFURIC
ACID AT SELECTED TIMES

| | | Time (mins) after | |
|---|---|---|---|
| Time (mins) after | Chloride | complete | Chloride |
| complete dissolution | content | dissolution of | content |
| of minocycline | (ppm)* | minocycline | (ppm) |
| Subsurface nitrogen | | Headspace nitrogen | |
| sparging | | sparging | |
| 0 | 1527 | 0 | 1725 |
| 30 | 1515 | 30 | 1673 |
| 60 | 1549 | 60 | 1941 |
| 90 | 1497 | 90 | 1674 |
| 180 | 1282 | 180 | 1750 |
| Vacuum | | No sparging | |
| 0 | 1852 | 0 | 2253 |
| 30 | —^{#} | 30 | 2269 |
| 60 | 1640 | 60 | 2224 |
| 90 | 1607 | 90 | 2349 |

*chloride content determined by IC.
^{#}not determined.

The experimental data suggests that nitrogen sparging has the ability to remove HCl from the system. Subsurface sparging showed a slightly better ability to remove HCl. Using a vacuum pull (300 torr) was comparable to nitrogen sparging in the headspace.

An additional experiment on 40 g scale with the identical setup describe above (250 ml round-bottom flask, 4 cm impeller length) but with the mixing speed increased to 500 rpm, the residual HCl was reduced to 623 ppm after 1 hour under vacuum (300 torr). This experiment undoubtedly shows mixing speed affects the removal of HCl as shown in Table 5. Following these observations, an experiment was performed on a 250 g scale in a 2-L round-bottom flask with impeller length 11 cm and vacuum applied at 300 torr. The mixing speed was 500 rpm. After 2 hr under vacuum, a sample of the minocycline in sulfuric acid showed the chloride content to be 138 ppm. Increasing the stirring rate will increase efficiency of HCl removal. In the experiments on 40 g scale, increasing the stirring rate 5-fold resulted in an approximately 2.5-fold decrease in HCl content. Vacuum range is 20 to 300 torr and speed is 100 to 500 rpm.

TABLE 5
EFFECT OF STIRRING AND VACUUM PRESSURE
ON CHLORIDE CONTENT

| | | | Chloride |
|---|---|---|---|
| Hold time | Stir rate | Pressure | content |
| (min) | (rpm) | (torr) | (ppm)* |
| 0 | 100 | 300 | 1548 |
| 60 | 500 | 300 | 623 |
| 60 | 500 | 50 | 146 |
| 60 | 500 | Full vac (<30) | 69 |

*reporting limit: 50 ppm

### Effect of Stirring and Vacuum on Hydrogen Chloride Removal

Further studies on the effect of stirring and effect on vacuum pressure on hydrogen chloride removal were conducted. The following data was collected on 40 g minocycline dissolved in 120 ml sulfuric acid in a 250-ml multi-neck round-bottom flask (diameter 8 cm), impeller length 4 cm as shown in Table 5 above, summarizes the chloride content of the solution on stirring rate and vacuum pressure.

Hydrogen chloride removal was dependent on the mixing rate and vacuum pressure. The chloride content was reduced by ˜60% (1548 ppm to 623 ppm) when the stir rate was increased from 100 rpm to 500 rpm as shown in Table 5_above while maintaining the vacuum pressure at 300 torr for 1 hr. When the vacuum pressure was decreased to 50 torr while maintaining the stir rate at 500 rpm, a further 77% decrease in chloride content was obtained. A further drop to full vacuum reduced the chloride content of the solution to 69 ppm. To efficiently remove residual hydrogen chloride upon dissolution of minocycline hydrochloride in sulfuric acid, the stirring rate should be relatively fast (500 rpm or above) as well as a vacuum pressure (300 torr or less) should be applied for a minimum of 1 hr. Full vacuum for the specific equipment used is an embodiment of this application. Vacuum range is 20 to 300 torr and speed is 100 to 500 rpm.

### Effect of Nitric Acid Addition Time on 9-nitrominocycline Purity

Three experiments were performed to examine the effect of nitric acid addition time on the purity of isolated 9-nitrominocycline. The experiments were performed on 50 g minocycline hydrochloride dissolved in 150 ml sulfuric acid in a 1-L ChemGlass cylindrical reactor (diameter 9.7 cm), impeller diameter 5 cm. The mixing speed was set at 500 rpm. The purity of the product at different addition times is presented in Table 6.

In all experiments, the starting material was consumed and the reaction went to completion, with less than 1.0% minocycline being found. Fast addition of nitric acid resulted in a higher level of 2 impurities (impurity B at rrt 0.59 and rrt 0.68) and a lower purity for 9-nitrominocycline as compared to the control reaction (Experiment 2). Extended addition of nitric acid did not change the purity profile when compared to the control reaction. The addition time had no significant effect on impurity A levels. The preferred addition time range is 100 min to 180 min.

TABLE 6
PURITY OF 9-NITROMINOCYCLINE ON
NITRIC ACID ADDITION TIME

| | | Purity 9- | | | |
|---|---|---|---|---|---|
| | | nitro-mino- | Impurity | Impurity | Impurity |
| Exper- | Addition | cycline | A | B (%) | B (%) |
| iment | time (min) | (%) | (%) | (rrt 0.59) | (rrt 0.68) |
| 1 | 30 | 55.29 | <0.1 | 11.29 | 9.90 |
| 2 | 100 | 70.58 | 0.11 | 7.95 | 6.85 |
| 3 | 360 | 71.80 | <0.1 | 7.68 | 6.54 |

### Effect of Mixing Rate and Location of Addition of Nitric Acid on Reaction Completion

To evaluate whether the mixing rate and addition of nitric acid at different locations within the reactor has any influence on reaction completion, three experiments were carried out with the location of addition of nitric acid as the variable. The location of addition were:
1. Addition of nitric acid sub-surface;
2. Addition of nitric acid above surface and between agitator and reactor wall;
3. Addition of nitric acid along reactor wall.

The following fixed parameters were established for these experiments. The reactor used was a 1-L cylindrical reactor (9.7 cm diameter). The mixing speed was set at 100 rpm and the ratio of the diameter of the reactor to the impeller (5 cm) was 2:1 in an attempt to mimic poor mixing. The nitric acid addition time was over 100 minutes.

In order to eliminate the effect of residual hydrogen chloride on nitration, a 250 g batch of minocycline sulfate was prepared and hydrogen chloride was removed by vacuum. The residual chloride content was 138 ppm. This batch was sub-divided by weight for the three reactions described as follows.

In one experiment on ˜83.0 g scale, when the nitric acid was added sub-surface between the center and wall of the reactor, the in-process test showed 0.16% unreacted minocycline starting material after 1.2 eq of nitric acid. This result shows that reaction completion can be achieved when nitric acid was added sub-surface even with slow mixing. Vacuum was not applied during nitric acid addition. Stirring rate was 100 rpm. This applies to the 3 experiments described here.

In the second experiment on ˜83.0 g scale (control reaction) where the nitric acid was added above surface and between the agitator and wall of the reactor, the in-process test showed 17% unreacted minocycline after 1.2 eq of nitric acid. An additional 0.3 eq of nitric acid (total 1.5 eq) showed 1.7% unreacted starting material. This result suggests that mixing rate is a parameter.

In the third experiment on ˜83.0 g scale where nitric acid was added along the reactor wall, the in-process test showed 28% unreacted minocycline starting material after adding 1.2 eq nitric acid and 9% unreacted after 1.5 eq. This result provides further indication that mixing rate plays a significant role in the nitration reaction. The poorer mixing along the reactor wall (2:1 ratio of reactor diameter to impeller diameter) compared to the mixing near the agitator resulted in an incomplete reaction. When the mixing rate was increased to 300 rpm, after 30 mins, unreacted starting material remained at 9%. After isolation and analysis, the product purity was low (56%) as expected and the starting minocycline was present at 4.8%. The excess nitric acid, however, did not increase the level of impurity B. The isolated material contained ˜8.5% impurity B.

When the second experiment described above was repeated on ˜50.0 g scale but with the mixing rate set at 500 rpm instead of 100 rpm during nitric acid addition, the reaction was complete (In-process control ˜0.34%) after 1.2 eq of nitric acid. In summary, the mixing rate should be increased (≧500 rpm) if above surface addition of nitric acid is implemented. Reaction completion can be achieved with slow mixing (100 rpm) if sub-surface addition is implemented.

### Effect of Holding Time Before Filtration on 9-nitrominocycline Purity

The effect of holding time before filtration and isolation of the 9-nitrominocycline on the purity profile was evaluated. Four experiments were performed at 0-10° C.:
1. Filtration of 9-nitrominocycline after 1 hr hold.
2. Filtration of 9-nitrominocycline after 18 hr hold.
3. Filtration of 9-nitrominocycline after 24 hr hold.
Filtration of 9-nitrominocycline after 48 hr hold.

The data in Table 7 shows the purity profile data collected from each of the experiments after washing the wet cake with IPA and drying. Data shows there is no notable difference in purity of the product isolated but the levels of impurity B are increased slightly as the reaction mixture was held for longer periods before filtration. It is preferable to hold for between 1 hr and 24 hr.

TABLE 7
EFFECT OF HOLDING TIME BEFORE FILTRATION ON
9-NITROMINOCYCLINE PURITY

| Hold time before | | | |
|---|---|---|---|
| filtration (hr) | Purity (%) | Impurity A (%) | Impurity B (%) |
| 1 | 74.8 | 0.3 | 5.6 |
| 18 | 76.1 | 0.2 | 6.2 |
| 24 | 76.2 | 0.2 | 6.4 |
| 48 | 71.8 | 0.2 | 6.6 |

### Conditions and Parameters that Affect Filtration

The effect of several parameters on the filtration of the nitration product was investigated: addition temperature, rate of addition, addition regime (direct and reverse), stirring rate, the quantity of sulfuric acid used and the quantity of antisolvent used.

In reverse addition experiments, where the reaction mixture is added to antisolvent (IPA/heptane), a higher addition temperature and lower acid quantity were found to favor faster filtration. In direct addition experiments, where the antisolvent is added to the reaction mixture, a higher addition temperature and a slower addition rate resulted in faster filtration. Overall, as further discussed below, the direct addition regime where the antisolvent is added to the reaction mixture resulted in a faster filtration rate.

### EXAMPLES

Addition time, antisolvent volumes, addition temperature, sulfuric acid quantity, stirring rate and the addition regimes (direct and reverse) were studied in a 50 mL automated multimax system.

Set 1—Experiments where the Reaction Mixture was Added to an Antisolvent.
Table 8 shows the studied variables and the lower/higher values of each.

TABLE 8
Variables for reverse addition experiments

| Variable | Low | High |
|---|---|---|
| Addition time, min | 30 | 180 |
| Addition temperature, C. | 10 | 32 |
| H2SO4, wt to wt of minocycline | 3.9 | 5.8 |
| HCl | | |
| Stirring rate, rpm | 200 | 800 |

Sixteen experiments were run the results of which are given in Table 9. Statistical analysis of the results was performed using Design Expert® software and it was found that for the reverse addition regime, higher addition temperature, faster addition of reaction mixture and lower sulfuric acid quantity result in faster filtration. The stirring rate showed a very weak influence on filtration rate.

TABLE 9
The results of sixteen experiments where the reaction mixture is added to
antisolvent. (Scale = 1 g) In the table, PSD stands for “particle size distribution”.

| | | | H2SO4, wt to wt | | Wash | total filt. + | |
|---|---|---|---|---|---|---|---|
| Stirring | Addition | Addition | of minocycline | Filtration | time, | wash, | |
| rate, rpm | rate, min | temp, C. | HCl | time, sec | sec | sec | PSD |
| 200 | 30 | 32 | 3.9 | 14 | 28 | 42 | DV[90] = 46 |
| | | | | | | | DV[50] = 23 |
| 200 | 180 | 32 | 3.9 | 15 | 34 | 49 | |
| 800 | 30 | 32 | 3.9 | 20 | 59 | 79 | |
| 200 | 30 | 10 | 3.9 | 50 | 70 | 120 | |
| 800 | 180 | 32 | 3.9 | 44 | 128 | 172 | |
| 800 | 30 | 10 | 3.9 | 55 | 140 | 195 | |
| 200 | 30 | 32 | 5.8 | 80 | 185 | 265 | |
| 800 | 30 | 32 | 5.8 | 80 | 190 | 270 | |
| 200 | 180 | 32 | 5.8 | 60 | 225 | 285 | |
| 800 | 180 | 32 | 5.8 | 60 | 240 | 300 | |
| 200 | 30 | 10 | 5.8 | 90 | 235 | 325 | |
| 800 | 30 | 10 | 5.8 | 120 | 255 | 375 | DV[90] = 36 |
| | | | | | | | DV[50] = 17 |
| 800 | 180 | 10 | 5.8 | 210 | 170 | 380 | |
| 200 | 180 | 10 | 3.9 | | | | |
| 200 | 180 | 10 | 5.8 | | | | |
| 800 | 180 | 10 | 3.9 | | | | |

Set 2—Experiments where an Antisolvent was Added to Reaction Mixture
Table 10 shows the studied variables and the lower/higher values of each.

TABLE 10
Variables for antisolvent addition experiments

| Variable | Level 1 | Level 2 |
|---|---|---|
| Addition time, min | 30 | 180 |
| IPA/Heptane volumes | 15 | 24 |
| Addition temperature | 10 | 32 |
| H2SO4, wt to wt of | 3.9 | 5.8 |
| minocycline HCl | | |
| Stirring rate, rpm | 200 | 800 |
| Precipitation regime | Adding reaction | Adding antisolvent to |
| | mixture to antisolvent | reaction mixture |

Sixteen experiments were run in which the results are given in Table 11. Statistical analysis of the results was performed using Design Expert® software and it was found that, for the direct antisolvent addition regime, higher addition temperature and slower addition of the reaction mixture were the main factors that result in faster filtration.

TABLE 11
The results of various experiments where antisolvent is added to the reaction mixture

| | Addition | | | H2SO4, | Filtration | Wash | total filt. + |
|---|---|---|---|---|---|---|---|
| | rate, | IPA/Heptane | Addition | wt to | time/scale, | time/scale, | wash/scale, |
| Scale, g | min | volumes | temperature | wt | sec | sec | sec |
| 3 | 180 | 15 | 32 | 3.9 | 2 | 1 | 3 |
| 2 | 180 | 15 | 32 | 5.8 | 3 | 3 | 6 |
| 2 | 180 | 24 | 32 | 5.8 | 4 | 3 | 6 |
| 2 | 180 | 24 | 32 | 3.9 | 4 | 3 | 7 |
| 2 | 30 | 24 | 32 | 3.9 | 4 | 5 | 9 |
| 2 | 30 | 24 | 32 | 5.8 | 6 | 3 | 9 |
| 3 | 30 | 15 | 32 | 3.9 | 3 | 6 | 9 |
| 3 | 30 | 15 | 32 | 5.8 | 4 | 6 | 10 |
| 2 | 180 | 24 | 10 | 3.9 | 7 | 7 | 14 |
| 3 | 180 | 15 | 10 | 3.9 | 10 | 6 | 16 |
| 3 | 180 | 15 | 10 | 5.8 | 15 | 5 | 20 |
| 2 | 180 | 24 | 10 | 5.8 | 15 | 7 | 22 |
| 3 | 30 | 15 | 10 | 5.8 | 8 | 13 | 22 |
| 2 | 30 | 24 | 10 | 5.8 | 18 | 18 | 35 |
| 2 | 30 | 24 | 10 | 3.9 | 105 | 55 | 160 |
| 3 | 30 | 15 | 10 | 3.9 | 107 | 170 | 277 |

The effect of the purity of the starting material on filtration was also studied. In a scale-up run with a starting material having purity 44% (Table 12, scale-up run 1) in which the reaction mixture added to the antisolvent, the filtration time required was 26 minutes due the viscosity of the slurry formed.

It is believed that this slow filtration may be primarily due to the low purity of the starting material. For comparison, a separate experiment (scale-up run 2) was performed where the starting material had a purity of 72% in which the reaction mixture was added to the antisolvent. The filtration time required was only 3 minutes. For further comparison, another experiment (scale-up run 3) was performed where the starting material had a purity of 65% in which the antisolvent was added to the reaction mixture. The filtration time required was only 4 seconds.

The filtration time decreases as particle size increases, as shown below in Table 12:

TABLE 12
Particle size distribution according to various scale-up runs

| | Scale-up run | Scale-up run | Scale-up run |
|---|---|---|---|
| | 1 (63 g) | 2 (33 g) | 3 (45 g) |
| D[v, 0.1] | 3.1 | 2.82 | 2.9 |
| D[v, 0.5] | 12.8 | 17.6 | 24.3 |
| D[v, 0.9] | 39.6 | 47.9 | 60.5 |
| Filtration time | 26 mins | 3 mins | 4 secs |

The results of Table 12 suggest that direct addition could improve the rapidity of filtration by resulting in the formation of larger particles. Accordingly, a non-limiting illustrative example of the steps that may be employed after completion of the nitration reaction is as follows:
adjusting the temperature of the reaction mixture to 0-40° C.; preferably 23-24° C.;
adding 5 to 20%, preferably 10%, of an antisolvent over 20 to 120 minutes, preferably 20-30 miunutes, to the reaction mixture, wherein the antisolvent is added from or through a container fitted with a jacket, wherein the the jacket temperature is adjusted to 0-40° C.;
adding the remainder of the antisolvent over 2-5 hrs, preferably 2-2.5 hrs, to the reaction mixture while maintaining the reaction mixture temperature in the range of 0-40° C., preferably 29-32° C.;
stirring the reaction mixture for 1 hr-24 hours, preferably 1 hour, at 0-40° C., preferably 29-32° C.;
cooling the reaction mixture to 0-40 C, preferably 23-25° C., wherein the temperature to which the reaction mixture is cooled is lower than the temperature at which the reaction mixture is stirred; and
filtering the reaction mixture.
   Large-Scale Nitration with Improved Reaction Conditions

To evaluate whether the above findings can be reproducible on larger scale, a nitration reaction was conducted on 500 g of minocycline in a 5-L jacketed cylindrical reactor. To ensure that complete success can be achieved at commercial scale, the reaction conditions and reactor vessel parameters defined in the commercial batches were closely emulated in scaled-down reactors. Described in Table 13 is a comparison of the reactor vessel specifications used in the nitration reaction on commercial batches vs. the parameters used in our scaled-down equipment.

The scaled down agitation speed was calculated based on the following geometric similarity mixing equation:

$${rpm}_{2} = {rpm}_{1} \times \sqrt[3]{( \frac{{V}_{2}}{{V}_{1}} ) {( \frac{{D}_{1}}{{D}_{2}} )}^{5}}$$

where
rpm₂=scaled down equipment agitator speed (rpm)
rpm₁=large-scale equipment agitator speed (rpm)
V₁=maximum volume (L) on large scale equipment
V₂=maximum volume (L) on scaled down equipment
D₁=diameter (mm) of agitator on large scale equipment
D₂=diameter (mm) of agitator on scaled-down equipment

The commercial supply batches were typically performed on 186 kg of minocycline starting material. The estimated maximum volume in the reactor was 700 L and for the scaled-down experiments, the maximum volume measured was 2 L on 500 g scale. The geometric similarity calculation is based on the assumption that reactor shape and size ratios are held equal.

In a 5-L ChemGlass jacketed cylindrical reactor, minocycline hydrochloride was added to and dissolved in concentrated sulfuric acid at 0-10° C. The nitrogen flow was set at 0.2 SCFH and agitation rate at 492-500 rpm. The addition took 1 hr 45 min. A vacuum was applied at 287-300 torr for 3 hr. The mixture was allowed to stand at 0-5° C. (50 rpm) for 71 hr followed by vacuum at 50 torr for 1 hr and stand for another 17 hr. Data in Table 14 summarizes the purity and chloride content at different sampling points. The starting material minocycline hydrochloride contained 6.8% HCl.

TABLE 13
REACTOR SPECIFICATIONS FOR
5-L NITRATION REACTION

| | RA3-109 | CG-1929-28* |
|---|---|---|
| Reactor volume capacity | 4220 | 5 |
| (L) | | |
| Reactor diameter (mm) | 1600 | 176 |
| Agitator diameter (mm) | 1290 | 135 |
| Agitator type | Anchor + turbine | Teflon paddle |
| | anti foam | (half-moon) |
| Baffles | 2 (180°)-radial, | 1 external (1 cm |
| | external | diameter)^{§} |
| Temperature probe | 700 mm from center | 0.25″ diameter, |
| position | | 48 mm from center |
| Temperature probe | Min volume 300 litres | Bottom of probe |
| | | situated at the 1-liter |
| | | mark of reactor |
| Agitation speed (rpm) | 74 | 500^{¥} |
| Nitric acid charge setup | Dip tube above surface | Dip tube 13 cm above |
| | | surface |
| HCl purge | Headspace, PTFE | Vacuum (50-300 torr) |
| | lining 3″ | |

*ChemGlass, Vineland, NJ.
^{§}Bottom of the baffle situated at 1-liter mark of the reactor.
^{¥}Calculated agitation speed was 452 rpm.

TABLE 14
PURITY AND CHLORIDE CONTENT OF
MINOCYCLINE IN SULFURIC ACID

| | | Minocycline purity | Chloride |
|---|---|---|---|
| Experiment | Sampling point | (Total impurity, %) | content (ppm) |
| 1 | Minocycline | — | 68000 |
| 2 | Before vacuum | 9.68 | 364 |
| 3 | After vacuum at | 9.95 | <50* |
| | 300 torr for 3 hr | | |
| 4 | After 65 hr hold at | 8.33 | <50 |
| | 0-5° C. | | |
| 5 | After vacuum at | 8.73 | <50 |
| | 50 torr for 1 hr and | | |
| | 17 hr hold | | |

*reporting limit: 50 ppm

Effective removal of HCl was achieved after 3 hr mixing (500 rpm) at 300 torr. After removing HCl from the system after sampling for HCl content (Experiment 4), the minocycline was nitrated. The agitation rate was set at 500 rpm and nitric acid was added over 100 mins via a dip tube situated 13 cm above the surface of the reaction mixture. The reaction was completed (starting material was undetected by HPLC) using 1.2 eq nitric acid. Minocycline was less than 1.0%. The cold reaction mixture was transferred over 1 hr to a mixture of IPA:heptane (13.7 L IPA, 1.65 L heptane) kept at 0-12° C. in a 20-L ChemGlass jacketed reactor. The precipitated product was mixed at 0-10° C. overnight, filtered, washed with IPA:heptane (3.225 L IPA, 0.55 L heptane) followed by IPA (3.6 L). The product was dried at 40-42° C. to provide 613 g (93% yield) of 9-nitrominocycline sulfate. This procedure described above was repeated in the demonstration batch (see further ahead).

The purity of the 9-nitrominocycline produced was 76.5%. This purity was comparable or superior to the purity obtained in typical commercial batches.

This would be expected given the supplemental vacuum removal of residual HCl before nitration in this experiment. This operation step led to a much cleaner 9-nitrominocycline material. Comparison of the purity profile data collected for this experiment (pre-demo 500 g) with typical batches is presented in Table 15.

TABLE 15
9-NITROMINOCYCLINE PURITY PROFILE

| | Area % | | | | | | | | | | | | |
|---|---|---|---|---|---|---|---|---|---|---|---|---|---|
| | HPLC | | | | | | | | | | | | |
| | Relative Retention Time | | | | | | | | | | | | |
| | Mino | | | | IMP B | IMP B | | | | Epi | 9-Nitro | IMP A | |
| Description | 0.25 | 0.29 | 0.37 | 0.44 | 0.59 | 0.68 | 0.78 | 0.88 | 0.93 | 0.97 | 1.00 | 1.09 | 1.15 |
| NO2 sulfate(oven dry) | 0.68 | <0.1 | 0.77 | 1.40 | 5.54 | 4.25 | 1.29 | 1.53 | 2.55 | 1.72 | 76.27 | <0.1 | 1.34 |
| NO2 sulfate (N2 dry) | 0.64 | <0.1 | 0.92 | 0.09 | 2.94 | 3.17 | 1.57 | 6.13 | 5.48 | 2.45 | 72.91 | 0.26 | 0.25 |
| NO2 HCl (oven dry) | 1.24 | <0.1 | 1.51 | 1.85 | 8.78 | 7.06 | 3.58 | 2.58 | 4.22 | 1.46 | 61.65 | 0.30 | 1.92 |
| NO2 HCl (N2 dry) | 1.22 | <0.1 | 1.44 | 1.74 | 10.23 | 7.05 | 3.91 | 2.08 | 2.91 | 1.33 | 61.33 | 0.34 | 2.48 |
| 30 min add, Cl unknown | 0.07 | <0.1 | 0.84 | 0.86 | 8.10 | 4.20 | 4.18 | 1.40 | 1.56 | 2.37 | 58.21 | 8.40 | 0.14 |
| 30 min add, Cl not control, | 0.04 | <0.1 | 0.88 | 1.01 | 8.05 | 4.50 | 3.22 | 7.55 | 4.26 | 1.21 | 59.87 | 8.20 | 0.48 |
| after IPA wash | | | | | | | | | | | | | |
| 6 h add, Cl not control | 0.11 | <0.1 | 1.28 | 0.68 | 8.49 | 5.41 | 4.06 | 2.08 | 1.96 | 2.40 | 56.16 | 7.23 | 0.17 |
| | 0.07 | <0.1 | 1.21 | 0.99 | 9.86 | 5.88 | 2.01 | 6.32 | 3.38 | 1.27 | 53.61 | 7.24 | 1.01 |
| | nd | <0.1 | 1.20 | 2.26 | 11.85 | 8.73 | 3.30 | 2.86 | 1.97 | 1.14 | 53.11 | 6.49 | 2.26 |
| | 0.05 | <0.1 | 1.25 | 1.76 | 12.21 | 9.40 | 3.80 | 2.27 | 1.97 | 1.10 | 52.97 | 6.36 | 3.03 |
| 70 min add (wash-100 vol) | 0.32 | 0.16 | 2.76 | 1.61 | 8.26 | 7.9 | 3.37 | 2.58 | 4.31 | 1.15 | 44.3 | 17.73 | 1.87 |
| 70 min add, yellow powder | 0.26 | <0.1 | 2.26 | 1.89 | 11.29 | 7.99 | 3.90 | 0.87 | 1.14 | 1.00 | 44.44 | 18.00 | 2.79 |
| (wash 8 vol) | | | | | | | | | | | | | |
| 70 min add/brown sticky | 0.31 | <0.1 | 2.20 | 2.29 | 7.15 | 5.26 | 3.19 | 4.92 | 2.14 | 1.30 | 44.62 | 18.80 | 0.19 |
| (wash 8 vol) | | | | | | | | | | | | | |
| nitration/on walls | 5.11 | 0.2 | 2.0 | 3.25 | 9.8 | 5.61 | 4.64 | 1.15 | 1.98 | 1.65 | 54.67 | 1.34 | 1.47 |
| | 5.15 | 0.39 | 2.12 | 2.02 | 10.07 | 5.96 | 4.75 | 1.40 | 1.95 | 1.59 | 53.84 | 1.16 | 2.15 |
| nitration/subsurface | <0.1 | <0.1 | 0.97 | 0.88 | 7.24 | 6.27 | 2.36 | 2.77 | 2.62 | 1.60 | 68.90 | 1.58 | 1.75 |
| | <0.1 | <0.1 | 1.06 | 2.14 | 6.8 | 6.06 | 2.41 | 2.96 | 2.72 | 1.60 | 68.37 | 1.29 | 0.48 |
| nitration/middle | 0.77 | 0.15 | 1.44 | 2.83 | 11.54 | 8.85 | 4.65 | 2.11 | 3.01 | 1.54 | 52.45 | 1.38 | 2.34 |
| | <0.1 | 0.06 | 1.56 | 3.09 | 10.14 | 9.0 | 4.73 | 2.28 | 3.62 | 1.42 | 53.74 | 0.99 | 1.77 |
| 30 min HNO3 addition | <0.1 | 0.06 | 1.2 | 4.13 | 11.29 | 9.9 | 3.49 | 3.59 | 4.77 | 1.46 | 55.29 | <0.1 | 0.61 |
| 6 h HNO3 addition | <0.1 | 0.07 | 1.07 | 2.09 | 7.68 | 6.54 | 2.22 | 1.06 | 1.72 | 1.62 | 71.80 | 0.11 | 1.05 |
| 1.st IPA wash (102-2)/ | 0.14 | <0.1 | 1.1 | 2.13 | 6.13 | 5.39 | 2.46 | 3.01 | 2.63 | 1.88 | 68.80 | 1.23 | <0.1 |
| subsurface | | | | | | | | | | | | | |
| 2.nd IPA wash (102-2)/ | 0.13 | <0.1 | 1.09 | 1.88 | 5.73 | 5.12 | 2.92 | 3.5 | 3.28 | 2.1 | 67.58 | 1.3 | <0.1 |
| subsurface | | | | | | | | | | | | | |
| 3.rd IPA wash (102-2)/ | 0.13 | <0.1 | 1.09 | 1.98 | 6.06 | 4.72 | 3.29 | 3.02 | 2.86 | 2.39 | 62.89 | 1.36 | <0.1 |
| subsurface | | | | | | | | | | | | | |
| 1.st IPA wash (101-2)/walls | 4.87 | <0.1 | 2.14 | 2.77 | 8.45 | 5.18 | 3.41 | 1.86 | 2.11 | 1.51 | 55.60 | 1.17 | <0.1 |
| 2.nd IPA wash (101-2)/walls | 4.9 | 0.17 | 2.24 | 1.95 | 6.79 | 4.41 | 4.06 | 3.99 | 4.05 | 1.76 | 54.43 | 0.98 | <0.1 |
| 3.rd IPA wash (101-2)/walls | 5.09 | <0.1 | 2.3 | 2.44 | 8.54 | 4.44 | 4.49 | 1.73 | 2.25 | 1.82 | 55.78 | 1.03 | <0.1 |
| 1.st IPA wash (104-2)/middle | 0.72 | <0.1 | 1.61 | 3.57 | 10.1 | 7.5 | 4.12 | 2.83 | 3.16 | 1.51 | 54.50 | 1.02 | <0.1 |
| 2.nd IPA wash (104-2)/middle | 0.69 | <0.1 | 1.69 | 2.74 | 8.24 | 6.57 | 5.25 | 5.06 | 5.37 | 1.62 | 52.52 | 1.06 | <0.1 |
| 3.rd IPA wash (104-2)/middle | 0.76 | <0.1 | 1.68 | 3.44 | 10.36 | 6.31 | 5.41 | 2.73 | 3.33 | 2.1 | 53.85 | 1.2 | <0.1 |
| 100 min HNO3 addition | <0.1 | <0.1 | 1.02 | 2.69 | 7.95 | 6.85 | 2.34 | 1.04 | 1.46 | 1.57 | 70.58 | <0.1 | 1.38 |
| | 0.8 | 1.31 | 3.21 | n/d | 4.91 | 4.57 | 3.64 | 5.34 | 6.19 | 2.08 | 48.65 | 11.3 | 0.64 |
| | 0.81 | 1.33 | 2.69 | n/d | 8.70 | 5.23 | 3.35 | 1.20 | 1.39 | 1.32 | 53.95 | 12.87 | 1.74 |
| 100 min addition + 10% H20 | <0.1 | <0.1 | 1.29 | 3.1 | 7.63 | 6.48 | 2.25 | 1.38 | 1.5 | 1.62 | 68.97 | 0.15 | 0.31 |
| 100 min addition/SM: Chloride | <0.1 | <0.1 | 0.8 | 3.3 | 8.12 | 6.33 | 2.12 | 1.37 | 2.14 | 1.86 | 69.12 | 0.3 | 0.19 |
| 68 ppm | | | | | | | | | | | | | |
| 100 min addition/SM: Chloride | 0.12 | <0.1 | 1.16 | 2.6 | 7.06 | 5.22 | 1.95 | 1.24 | 1.65 | 1.82 | 65.91 | 5.87 | <0.1 |
| 482 ppm | | | | | | | | | | | | | |
| 100 min addition/SM: Chloride | <0.1 | 0.20 | 3.14 | 1.75 | 6.7 | 5.62 | 1.54 | 0.72 | 1.16 | 1.18 | 47.96 | 23.93 | 1.07 |
| 1332 ppm | | | | | | | | | | | | | |
| Predemo 500 g | <0.1 | <0.1 | 0.85 | 2.45 | 6.00 | 5.11 | 1.27 | 0.84 | 1.22 | 1.76 | 76.50 | 0.24 | 0.48 |
| Demo: filtrated 1 h after quench | <0.1 | <0.1 | 0.78 | 2.41 | 5.6 | 4.98 | 1.41 | 1.28 | n/d | 1.96 | 74.80 | 0.3 | 0.26 |
| Demo: filtrated 18 h after | <0.1 | <0.1 | 0.78 | 2.37 | 6.25 | 5.22 | 1.1 | 0.85 | 1.20 | 1.74 | 76.10 | 0.25 | 0.82 |
| quench(Batch) | | | | | | | | | | | | | |
| Demo: filtrated 24 h after | <0.1 | n/d^{a} | 0.77 | 2.62 | 6.4 | 4.97 | 1.31 | 0.83 | n/d | 1.86 | 76.20 | 0.20 | 0.46 |
| quench | | | | | | | | | | | | | |
| Demo: filtrated 48 h after | <0.1 | n/d | 0.77 | 2.80 | 6.60 | 4.89 | 1.38 | 0.60 | n/d | 1.92 | 71.80 | 0.20 | 0.23 |
| quench | | | | | | | | | | | | | |
| 3.5 eq HNO3 + HCl (g), | | | | | 12.7 | | | | | | | 14.70 | |
| (Impurity A, B) | | | | | | | | | | | | | |
| Effect of air, light 48 h (SM: | 0.56 | 0.02 | 2.11 | 0.13 | 0.22 | 4.88 | 2.69 | 11.24 | 8.63 | 5.03 | 47.05 | 0.63 | 0.05 |
| 39762-104-2) | | | | | | | | | | | | | |
| Commercial | 0.72 | n/a^{b} | n/a | n/a | n/a | 7.10 | n/a | n/a | n/a | 2.09 | 73.83 | 4.35 | 1.93 |
| Batches | 0.39 | n/a | n/a | n/a | n/a | 7.30 | n/a | n/a | n/a | 1.61 | 80.67 | n/a | 2.27 |
| | 0.44 | n/a | n/a | n/a | n/a | 7.28 | n/a | n/a | n/a | 1.88 | 79.67 | 2.00 | 2.50 |
| | 0.13 | n/a | n/a | n/a | 6.56 | n/a | n/a | n/a | n/a | 1.46 | 65.98 | 17.49 | 2.13 |
| | 0.37 | n/a | n/a | n/a | 8.15 | n/a | 1.09 | n/a | n/a | 1.78 | 79.21 | n/a | 2.14 |
| | 0.36 | n/a | n/a | n/a | n/a | 7.67 | 4.75 | n/a | n/a | 12.37 | 70.10 | n/a | 0.64 |
| | n/d | n/a | n/a | n/a | n/a | 20.20 | 0.93 | n/a | n/a | 1.39 | 69.15 | n/a | n/a |
| | 0.54 | 0.20 | n/c | n/c | n/a | 7.49 | n/a | n/a | n/a | 1.72 | 67.95 | n/a | n/a |
| | 0.10 | 0.11 | 0.27 | 0.75 | n/a | 20.19 | n/a | n/a | n/a | 2.17 | 63.85 | n/a | n/a |
| | 0.04 | 0.04 | 0.42 | 0.83 | n/a | 15.10 | n/a | n/a | n/a | 1.46 | 73.46 | n/a | n/a |
| | 0.08 | 0.11 | 0.11 | 0.63 | n/a | 18.12 | n/a | n/a | n/a | 1.65 | 66.47 | n/a | n/a |
| | 0.07 | n/a | 0.36 | 0.64 | n/a | 10.24 | n/a | n/a | n/a | 1.43 | 70.78 | n/a | n/a |

^{a}not detected;
* not available

### Demonstration Batch on Nitration of Minocycline Hydrochloride

To further validate our process, we performed the nitration reaction under the same scaled-down conditions described above using minocycline hydrochloride from commercial sources. Based on scientific data gathered from previous reactions, we modified slightly the HCl removal protocol. The procedure was simplified by applying vacuum at 50 torr for up to 3 hr before nitration. A summary of the chloride content at several times before nitration is presented in Table 16.

TABLE 16
CHLORIDE CONTENT OF MINOCYCLINE
IN SULFURIC ACID

| Sampling point | Chloride content (ppm) |
|---|---|
| Before vacuum | 1526 |
| After vacuum at 50 | 51 |
| torr for 1 hr | |
| After vacuum at 50 | <50* |
| torr for 2 hr | |
| After vacuum at 50 | <50 |
| torr for 3 hr | |
| After no vacuum, | 201^{¥} |
| o/n,^{§} 50 rpm | |

*reporting limit: 50 ppm;
^{§}o/n = overnight;
^{¥}accuracy of ± 100 ppm.

The chloride content, after holding overnight at 5° C. with no vacuum was 201 ppm owing to the accuracy (±100 ppm) of the determinations. The nitration reaction was completed using 1.2 eq nitric acid. During the addition of nitric acid over 100 min, the batch temperature was maintained at ˜6° C. while the jacket set temperature was at 10° C. It is interesting to note that when ˜1.0 eq of nitric acid was added based on volume, the batch temperature started to decrease which was indicative of the end of the reaction exotherm. Following precipitation from IPA:heptane and filtration, 9-nitrominocycline was obtained in 93% yield (minocycline less than 1%).

The purity profile presented in Table 15 was very similar to the first 500 g batch described above.

### Hydrogenation of 9-nitrominocycline

### Organic Impurities in 9-nitrominocycline

### Preparation of Enriched Impurity A

To study the effect of impurity A on hydrogenation we attempted to prepare an enriched sample of impurity A. The first attempt was by adding sodium chloride to the reaction mixture during nitration to generate HCl in situ and this led to higher levels of impurity A (∫17 area %). While not being bound by theory, impurity A has been identified as X-chloro-X—H₂O-9-nitrominocycline based on LCMS analysis. The molecular weight was found to be MW 554. The location of chlorine atom and water component on the molecule has not been determined conclusively.

We have found that impurity A could be prepared up to ˜70-75% purity by following a typical nitration of minocycline hydrochloride but under a stream of HCl gas. Hydrogen chloride gas when reacted with nitric acid produces chlorine gas that can chlorinate the minocycline before nitration and/or chlorinating 9-nitrominocycline producing impurity A. Nitration is completed using 2.4 eq of nitric acid. A 56 g batch was prepared.

One observed physical property of impurity A was the very hygroscopic nature. The isolated material was a brown colored solid when filtered onto a Büchner funnel. Drying at 23° C. overnight in vacuum oven, the material remains a brown solid. Further drying at 40° C. under vacuum resulted in evaporation of water. It is apparent that this impurity undergoes rapid dehydration or loss of surface water. On standing in air, the impurity darkens and forms a gummy substance.

A dynamic vapor sorption (DVS) study on this impurity shows that the compound picks up to 50% by weight of water at RH 90%. As a comparison, we ran the DVS on 9-nitrominocycline. This intermediate picks up to 40% by weight of water at RH 90%.

### Preparation of Enriched Imputity B

To study the effect of impurity B on hydrogenation, a batch of 9-nitrominocycline was prepared with an enriched sample of impurity B. Impurity B appears as a double peak on the analytical HPLC. While not being bound by theory, impurity B has been identified as a mixture of two (2) over-nitrated 9-nitrominocyclines. The location of the additional ‘nitro’ groups on the molecule has not been determined. Further, while not being bound by theory, the locations of these nitro groups are attached onto the hydroxyl groups of 9-nitrominocycline to form the nitro ester(s) of 9-nitrominocycline. The molecular weight was found to be MW 547 by LCMS indicative of the addition of one nitro group onto the molecule.

We have found that impurity B could be prepared up to ˜25% purity by adding excess nitric acid during nitration of minocycline hydrochloride. Nitration was completed using up to 3.5 eq of nitric acid.

Impurity B behaves similarly to impurity A in that on standing in air, the compound darkens and forms a gummy dark substance.

### Effect of Solvent Mixture

Based on the preliminary observations (Table 1), solvent (Factor 3) plays a role in the ability of the hydrogenation reaction to reach completion. In 99:1 methanol:water only (run 1), the reaction stalls with 51% starting material remaining whereas in 80:20 water:methanol mixture only (run 2), the reaction goes to completion.

For further confirmation that solvent mixture is a parameter, two more batches of lower quality 9-nitrominocycline were subjected to the same DOE hydrogenation screen. These additional experiments expanded the scope of the DOE screen to effectively include purity as a potential parameter. The results were consistent with the results observed earlier as presented in Table 17. As presented, the reaction goes to completion in 80:20 water:methanol mixture and was incomplete in 99:1 methanol:water.

TABLE 17
2-LEVEL 3-FACTOR DOE OF THE
HYDROGENATION OF 9-NITROMINOCYCLINE

| | Factor 1 | | | | |
|---|---|---|---|---|---|
| | Impurity A | Factor 2 | Factor 3 | | |
| Run | (%) | IPA (%) | Solvent | SM (%)^{a} | SM (%)^{b} |
| 1 | 0.00 | 0.00 | 99% MeOH | 53 | 96 |
| 2 | 0.00 | 0.00 | 80% H₂O | nd* | 99 |
| 3 | 0.00 | 50.00 | 99% MeOH | 61 | 87 |
| 4 | 0.00 | 50.00 | 80% H₂O | nd | 99 |
| 5 | 10.00 | 0.00 | 99% MeOH | 52 | 100 |
| 6 | 10.00 | 0.00 | 80% H₂O | 21 | 100 |
| 7 | 10.00 | 50.00 | 99% MeOH | 63 | 93 |
| 8 | 10.00 | 50.00 | 80% H₂O | 2 | 100 |

*nd = not detected.
^{a}Purity: 52.5%, Impurity A: 1.4%, Impurity B 11.5%.
^{b}Purity: 48.7%, Impurity A: 11.3%, Impurity B 4.9%.

No reaction occurred for any of the reactions in the DOE screen when the purity of the starting material was 48.7%. Statistical evaluation of the data revealed that the solvent and purity interactions are significant which implies that purity of the starting material could be a parameter in the outcome of the hydrogenation. In one experiment, the purity of 9-nitrominocycline was 48.7% and this low purity could explain why the hydrogenation did not proceed. These observations, however, are in contrast to the failed hydrogenations observed at commercial scale with batches of 9-nitrominocycline in the 63.8 to 71.8% purity range. A further investigation on the level of residual solvent IPA in the 9-nitrominocycline was initiated.

The above DOE screen was replicated to test the validity our experimental protocol and observations. 9-Nitrominocycline with a purity of 53.9% was used for the study. When the hydrogenation DOE screen was conducted, the results of the DOE screen duplicated those observed previously. In all 8 experiments, no reaction occurred. This verified that our experimental technique is valid and also strengthens our proposed conclusion that both purity of starting material and solvent mixture have compelling interactions that influence hydrogenation reaction completion.

The factors that strongly and directly influence the hydrogenation are the solvent mixture, purity of starting material and the interaction of the two components. The solvent mixture and purity of starting material are thus considered parameters that affect the outcome of the hydrogenation.

### Effect of Residual Isopropanol (IPA) Solvent in 9-nitrominocycline

Under the hydrogenation conditions specified, doping the starting material with IPA up to 50% by weight, the reaction goes to completion in 80:20 water:methanol mixture as seen in Table 1, run 4, but not in 99:1 methanol:water as seen in Table 1, run 3. In two different batches in 99:1 methanol:water, there were 61% unreacted 9-nitrominocycline. The addition of 50% by weight IPA further inhibits hydrogenation compared to the non-doped reaction in 99:1 methanol:water as seen in Table 1, runs 1 and 3. These results suggest that IPA is not a parameter in the hydrogenation reaction in 80:20 water:methanol but could play a modest role in 99:1 methanol:water.

In 80:20 water:methanol, doping with 50% IPA and 10% impurity A show 2% starting material remaining as seen in Table 17, run 8. By monitoring the hydrogen consumption during the reaction, run 8 shows that if the hydrogenation was held beyond the allotted 5 hr reaction time, the reaction could potentially go to completion. The hydrogen uptake shows no leveling off after 5 hr. The experiment in run 8 was repeated and the reaction was complete after 6-7 hr, thus confirming the model.

In preliminary experiments on small scale on the Endeavour, an increase of IPA from 50 up to 200% (w/w) vs 9-nitrominocycline, the reaction slows down in 99:1 methanol:water whereas no effects were observed in 80:20 water:methanol.

Based on our experimental observations, residual IPA in the starting material has no important affect on reaction completion from 0 to 50% loading in 80:20 water:methanol. IPA has a moderate effect on reaction completion in 99:1 methanol:water.

### Solubility of 9-nitrominocycline and 9-aminominocycline Sulfate

An investigation by examining the solubility characteristics of 9-nitrominocycline and 9-aminominocycline sulfate in the presence of various amounts of IPA to gauge whether the starting material and/or product could in fact precipitate out in the reaction solvent was initiated.

The solubility of 9-nitrominocycline is fairly high in 80:20 water:methanol and doping the material with IPA does not change its solubility significantly as shown in Table 18. In 80:20 water:methanol, 9-nitrominocycline is not likely to precipitate.

TABLE 18
SOLUBILITY OF 9-NITROMINOCYCLINE

| | | Solubility at RT |
|---|---|---|
| Exp. No | Solvent | (mg/ml) |
| | MeOH:water (99:1, v:v) | |
| 1 | +0% wt/wt IPA/(MeOH:water) | >466 |
| 2 | +4% wt/wt IPA/(MeOH:water) | 129 |
| 3 | +20% wt/wt IPA/(MeOH:water) | 68 |
| 4 | +35% wt/wt IPA/(MeOH:water) | 30 |
| 5 | +50% wt/wt IPA/(MeOH:water) | 23 |
| 6 | +0% wt/wt IPA/(MeOH:water) | >421 |
| 7 | +4% wt/wt IPA/(MeOH:water) | >426 |
| 8 | +20% wt/wt IPA/(MeOH:water) | >453 |
| 9 | +35% wt/wt IPA/(MeOH:water) | >303 |
| 10 | +50% wt/wt IPA/(MeOH:water) | 333 |

In 99:1 methanol:water, however, the solubility of 9-nitrominocycline decreases as IPA is added. With high levels of residual solvent in the 9-nitrominocycline, the hydrogenation reaction in 99:1 methanol:water may result in precipitation of 9-nitrominocycline.

The data also shows 9-aminominocycline sulfate has somewhat low solubility in 99:1 methanol:water as shown in TABLE 19.

TABLE 19
SOLUBILITY OF 9-AMINOMINOCYCLINE

| | | Solubility at |
|---|---|---|
| Exp. No | Solvent | RT (mg/ml) |
| | MeOH:water (99.1, v:v) | |
| 1 | +0% wt/wt IPA/(MeOH:water) | 14 |
| 2 | +4% wt/wt IPA/(MeOH:water) | 16 |
| 3 | +20% wt/wt IPA/(MeOH:water) | 12 |
| 4 | +35% wt/wt IPA/(MeOH:water) | 9 |
| 5 | +50% wt/wt IPA/(MeOH:water) | 7 |
| 6 | +0% wt/wt IPA/(MeOH:water) | high |
| 7 | +4% wt/wt IPA/(MeOH:water) | high |
| 8 | +20% wt/wt IPA/(MeOH:water) | 243 |
| 9 | +35% wt/wt IPA/(MeOH:water) | 103 |
| 10 | +50% wt/wt IPA/(MeOH:water) | 58 |
| 11 | IPA Only | 1 |

In 80:20 water:methanol, 9-aminominocycline sulfate has high solubility. Doping the material with increasing amounts of IPA decreases the solubility but still sufficiently high (58 mg/ml) with 50% wt/wt IPA added. In 80:20 water:methanol, 9-aminominocycline is not likely to precipitate out but in the presence of sufficiently high levels of residual IPA (>50%), it could cause precipitation. 9-Aminominocycline sulfate has somewhat low solubility in 99:1 methanol:water (shown in Table 19). Lacing the material with increasing amounts of IPA decreases the solubility. The hydrogenation reaction in 99:1 methanol:water could result in precipitation of 9-aminominocycline.

Both 9-nitrominocycline and 9-aminominocycline sulfate may precipitate in 99:1 methanol:water during hydrogenation if high levels of IPA is present. Observations that in the hydrogenations conducted in 99:1 methanol:water, the hydrogen uptake traces show a plateau at ˜50% completion and a gummy substance is deposited at the bottom of the reactor. The effect of adding sulfuric acid has not been examined. No deposit was observed in 80:20 water:methanol. While not being bound by theory, poisoning of the catalyst is a possibility.

### Effect of Impurity A on Hydrogenation

At 2 ml scale using the configuration and set-up described above (Endeavour), spiking the starting material with impurity A up to 10%, the reaction goes to completion in 80:20 water:methanol mixture but not in 99:1 methanol:water as shown in Table 1, run 5 and 6. This is an indication that impurity A is not a parameter in the hydrogenation reaction in both solvent systems and that the solvent itself is the contributor to the incomplete hydrogenation. In addition, the level of unreacted starting material remaining (49%) in 99:1 methanol:water does not change compared to the unspiked starting material as shown in Table 1 in run 1 and 5. Moreover, it was observed that impurity A was converted to the desired-9-aminominocycline upon hydrogenation. While doping batch with 10% impurity A show 21% starting material remaining as shown in Table 17, run 6) and 2% starting material remaining when doped with 10% impurity A and 50% IPA as seen in Table 17, run 8, these results were considered not significant based on a statistical analysis.

Therefore, a replicate study on this reaction was conducted and confirmed the reaction indeed went to completion. In addition, by monitoring the hydrogen consumption during the reaction as seen in both runs 6 and 8 showed no leveling off indicating that the reaction could potentially go to completion if left longer. The experiments in run 6 and 8 were therefore repeated. The reactions were complete after 6-7 hr.

As shown, impurity A has no significant affect on reaction completion from 0 to 10% loading in either 80:20 water:methanol or 99:1 methanol:water mixture in the hydrogenation.

### Reduction of Impurity A to 9-aminominocycline

Impurity A contains additional chlorine and a water molecule in 9-nitrominocycline (MW 554 as free base). Under the hydrogenation conditions in the presence of palladium catalyst impurity A is converted to 9-nitrominocycline. The first step would be loss of chlorine followed by loss of water to give 9-nitrominocyline. 9-Nitrominocycline, in turn, gets further reduced to 9-aminominocycline. This was evident when we followed the reaction by UPLC-MS. The MS analysis detected peaks that corresponded to molecular weights of 502, 519 and 473. These peaks are consistent with the loss of chlorine from impurity A (MW 519). Loss of water produces 9-nitrominocycline (MW 502). Further hydrogenation of 9-nitrominocycline led to 9-aminominocycline (MW 473).

### Hydrogenation Scale-Up

To confirm the DOE screening experiments done on small scale, reactions were carried out on 20 g scale in a 300 ml Parr reactor in both solvent conditions as shown in Table 20.

TABLE 20
SCALE-UP OF DOE EXPERIMENTS AT 20 G SCALE

| | Solvent | 9-nitro | 9-Amino | |
|---|---|---|---|---|
| Run^{a} | ratio | (%) | (%) | Comments |
| 1^{b} | MeOH/H₂O | 62 | 17 | 21% of Imp |
| | (99/1) | | | A remain |
| 2^{b} | H₂O/MeOH | 52 | nd* | 48% of IMP |
| | (80/20) | | | A remain |
| 3 | MeOH/H₂O | 60 | 40 | b |
| | (99/1) | | | |
| 4 | H₂/MeOH | Nd | 100 | b |
| | (80/20) | | (+ epimer) | |
| 5 | MeOH/H₂O | 60 | 40 | Appearance |
| | (99/1) | | | of solids |
| 6 | H₂O/MeOH | Nd | 100 | |
| | (80/20) | | (+ epimer) | |
| 7 | MeOH/H₂O | — | | |
| | (99/1) | | | |
| 8 | H₂O/MeOH | Nd | 100 | |
| | (80/20) | | (+ epimer) | |
| 9 | MeOH/H₂O | — | | |
| | (99/1) | | | |
| 10 | H₂O/MeOH | Nd | | |
| | (80/20) | | | |

*nd = not detected.
^{a}Conditions: 300 ml Parr reactor, 5% wt 5% Pd/C (50% wet), 70 psi H₂, 6.5 vols of solvent, 950 rpm, duration 5 hrs.
^{b}2.5% wt 5% Pd/C (50% wet)

A 300 ml Parr reactor was configured and setup, as shown in Table 21 to emulate large-scale hydrogenation vessels.

TABLE 21
REACTOR SPECIFICATIONS FOR 300 ML
HYDROGENATION REACTION

| | HS3-04^{#} | Parr 452HC* |
|---|---|---|
| Reactor volume | 4000 | 0.30 |
| capacity (L) | | |
| Reactor diameter (mm) | 1500 | 63 |
| Agitator diameter | 505 | 35 |
| (mm) | | |
| Agitator type | Double stage turbine | Double stage turbine |
| | (90°) + turbine anti foam | (45°) pitched blade |
| Baffles | 3 (605 mm from center) | 2 external (6.5 mm |
| | | diameter, 1.5 cm |
| | | from center)^{§} |
| Temperature probe | On one of the baffles | 3 mm diameter, |
| position | | 2.25 mm from center |
| Temperature probe | Min volume 500 litres | Bottom of probe |
| | | situated 8 mm from |
| | | bottom of reactor |
| Agitation speed (rpm) | 185 | 950^{¥} |

^{#} Commercial large scale hydrogenation vessels.
*Parr Instrument, Moline, Illinois.
^{§}bottom of the baffle situated 8 mm from bottom of the reactor.
^{¥}maximum agitation speed attainable on instrument. Calculated agitation speed based on geometric similarity equation was 700-800 rpm.

The results at 300 ml scale duplicates very well with those obtained at 2 ml scale using the Endeavour. In 99:1 methanol:water, the reaction stalls at 60% starting material whereas the reaction went to completion (SM<1.0%) in 80:20 water:methanol as seen in Table 20 runs 4, 6, 8, 10. The reaction in 80:20 water:methanol was performed on three different batches of 9-nitrominocycline and in all three cases, the reaction went to completion. The 20 g reaction with enriched impurity A (75% enrichment) did not proceed to completion in either solvent system as shown in Table 20 runs 1, 2. Based on these and the smaller scale experiments, we suggest to have a specification of up to 10% impurity A in 9-nitrominocycline as shown in Table 1 in run 6. The typical levels of impurity A in our nitration experiments were no more than (NMT) 2.0%.

Preliminary attempts to hydrogenate 9-nitrominocycline enriched with ˜25% impurity B were unsuccessful in both 99:1 methanol:water and 80:20 water:methanol solvent mixtures.

### Effect of Residual Mother Liquors on Hydrogenation

The effect of residual mother liquors in the 9-nitrominocycline starting material on hydrogenation using the Endeavour was studied. The approach taken was to dose the vacuum dried 9-nitrominocycline with mother liquors from the nitration reaction before hydrogenation and observing the effect. The mother liquors contained sulfuric acid, IPA and heptanes as main components. Two studies were conducted. The first with mother liquors dosed as is and the second with mother liquors stripped of IPA and heptanes before dosing.

When the mother liquors used were IPA and heptanes-free, the hydrogenation reaction proceeded to completion in both 99:1 methanol:water and 80:20 water:methanol in the presence of mother liquors up to 100% by weight. The same conclusion can be made when mother liquors were not removed of IPA and heptanes.

### Effect of Sulfuric Acid on Hydrogenation

The effect of sulfuric acid on hydrogenation is presented in Table 22, which summarizes the results obtained when 9-nitrominocycline was dosed with 10% and up to 50% by weight sulfuric acid before hydrogenation. Reactions were conducted in the Endeavour hydrogenator on 2 ml scale.

In both solvent systems, the hydrogenation went to completion. While it was observed that the reaction in 99:1 methanol:water was incomplete without sulfuric acid as in Table 1, run 1 we observed reaction completion in this study when sulfuric acid was intentionally added to the same starting material (Table 22, Experiment 1). It is clear that residual sulfuric acid is a parameter in the hydrogenation reaction in 99:1 methanol:water. Isolated 9-nitrominocycline batches contain residual sulfuric acid and the hydrogenation would proceed to completion. With little or no residual sulfuric acid present, we would expect reaction incompletion as shown in our DOE study.

Analysis of one batch of 9-nitrominocycline showed sulfate content to be 25%. This level is lower than the theoretical amount of 28% that is representative of 9-nitrominocycline as a disulfate salt. The bounded sulfates would not likely participate or have influence in the hydrogenation. The determination of sulfates by our analytical methods, however, do not distinguish between bounded or unbounded/residual sulfuric acid.

We repeated the hydrogenation on three more batches in 99:1 methanol:water and in each case, the reaction went to completion as shown in Table 22 (Experiments 2, 3 and 4). A 20 g scale-up hydrogenation was performed on the demonstration batch of 9-nitrominocycline and it also went to completion in 99:1 methanol:water.

In 80:20 water:methanol, we observe the reaction proceeds to completion in the presence or absence of sulfuric acid, thus, indicating sulfuric acid is not a critical parameter in 80:20 methanol:water.

In summary, sulfuric acid appears to be a critical parameter when the hydrogenation is conducted using the reaction conditions (99:1 methanol:water). Sulfuric acid does not appear to be a critical parameter using the conditions (80:20 water:methanol).

TABLE 22
EFFECT OF SULFURIC ACID ON HYDROGENATION

| Experiment | H₂SO₄ spiking^{d} | Solvent^{a} | % SM^{b} |
|---|---|---|---|
| 1 | 50% wt | MeOH/H₂O | 0.3^{c} |
| | | (99/1 v/v) | |
| | | H₂O/MeOH | 0.3^{c} |
| | | (8/2 v/v) | |
| | 10% wt | MeOH/H₂O | nd*,^{c} |
| | | (99/1 v/v) | |
| | | H₂O/MeOH | nd^{c} |
| | | (8/2 v/v) | |
| 2 | 10% wt | MeOH/H₂O | nd^{c} |
| | | (99/1 v/v) | |
| 3 | 10% wt | MeOH/H₂O | nd^{c} |
| | | (99/1 v/v) | |
| 4 | 10% wt | MeOH/H₂O | 0.8^{c} |
| | | (99/1 v/v) | |

*nd = not detected.
^{a}Conditions: 5% wt 5% Pd/C (50% wet), 70 psi H₂, 6.5 vols of solvent, 500 rpm, duration 5 hrs, 25° C., solvent 2 ml.
^{b}9-aminominocycline epimer not considered into the SM % calculation.
^{c}9-aminominocycline epimer detected.
^{d}Concentrated sulfuric acid (66° Be).

### Demonstration Batch on Hydrogenation of 9-nitrominocycline Sulfate

To confirm and validate the suitability of the 9-nitrominocycline obtained by the ‘modified’ process and to confirm that our scale-down parameters were valid, we performed the hydrogenation reaction under scaled-down conditions using 9-nitrominocycline disulfate obtained from the nitration demonstration batch. The 2-gallon Parr reactor was configured and setup so as to emulate large-scale hydrogenation vessels.

The commercial supply batches were typically performed on 245 kg of 9-nitrominocycline starting material. The estimated maximum volume in the reactor was 1600 L and for the scaled-down experiments, the maximum volume measured was 3 L on 400 g scale. The geometric similarity calculation is based on the assumption that reactor shape and size ratios are held equal.

In a 2-gallon stirred pressure reactor (Parr 455SS) equipped with double stage pitched blade style impellers (9.9 cm diameter), baffle and cooling coil, 5% palladium on charcoal 50% water wet (20 g) and 9-nitrominocycline sulfate (400 g) were charged. The pressure reactor was purged three times with nitrogen. Methanol (412 g) and purified water (2.1 kg) were charged using nitrogen pressure. The agitation rate was set to 345-355 rpm and the reaction mixture was cooled to 5-10° C. The chilled reaction mixture was hydrogenated under 70 psi of hydrogen for 10 hours. The completion of the reaction was monitored by HPLC (SM %<0.5%). The catalyst was filtered through 0.2 μm cartridge (Pall VFTR200-04M3S). The reactor, the lines and the filter were rinsed with cooled (0-10° C.) purified water (490 g). The clarified solution was transferred using vacuum into a 5-L jacketed cylindrical reactor equipped with a pitched blade style impeller (120 mm diameter), thermocouple and nitrogen inlet. The temperature of the solution was adjusted to 0-5° C. Sodium sulfite (0.24 g) was added followed by HCl reagent (267.8 g) added over 15 min. The pH was 0.9. The pH was adjusted to 3.8 to 4.2 using ammonium hydroxide 28% (254.4 g). During this pH adjustment, the temperature was kept below 10° C. The mixture was held for 2 hours at 0-10° C. The solids were filtered on a Büchner funnel (20 cm diameter) and washed with cooled purified water (245 g, adjusted to pH 3.8-4.2) and acetone (579 g). The product was dried under vacuum at 40-45° C. until loss on drying (LOD) was ≦7%. The process provided 117 g in two crops (80 g+37 g, 43% overall yield from minocycline) of 9-aminominocycline HCl. The second crop material was recovered after allowing the mother liquors to stand overnight in the refrigerator. The isolated yield was within the expected yield range (expected yields 38-62% from minocycline). The lower yield could be a result of the scale-down effect.

The purity of the 9-aminominocycline produced was 95.9% (first crop) and 96.3% (second crop). This purity achieved was comparable to the purity obtained in typical manufacturing batches. In summary, we have shown that hydrogenation can be achieved successfully and reproducibly in 80:20 water:methanol mixture using the improved reaction conditions for the nitration step.

### Nitration

The parameters identified in the nitration of minocycline were residual gaseous hydrogen chloride during dissolution of minocycline and mixing rate during nitric acid addition. Strong evidence as shown in the experiments herein that if residual HCl is below the reporting limit of NMT 50 ppm (control space), good quality of 9-nitrominocycline will be obtained. Further evidence that if the residual HCl is NMT 482 ppm (design space limit), good quality of 9-nitrominocycline will still be achieved. We have support that the mixing rate during nitration (NLT 500 rpm) will provide good quality 9-nitrominocycline.

### Hydrogenation

The design space for the three parameters in the hydrogenation were established based on experimental data collected in this study. The parameters identified were purity of 9-nitrominocycline, solvent for hydrogenation and sulfuric acid. From this knowledge laboratory experiments showed that the purity of 9-nitrominocycline in the hydrogenation could inhibit reaction completion. The solvent plays a role in hydrogenation. Substantial evidence shows that hydrogenation will proceed in 80:20 water:methanol. We have shown that sulfuric acid is also a attribute and that in the presence of NLT 10% hydrogenation will go to completion.

Evaluation of parameters suggests residual gaseous hydrogen chloride during dissolution of minocycline and mixing rate during nitric acid addition are key elements in the nitration. Factors that affect the hydrogenation of 9-nitrominocycline to 9-aminominocycline were related to quality of 9-nitrominocycline, reaction solvent and residual sulfuric acid.

### Example of Nitration

### Preparation of 9-nitrominocycline Sulfate

In a 5-L jacketed cylindrical reactor, minocycline hydrochloride dihydrate (500 g, 0.94 mole) was added to and dissolved in concentrated sulfuric acid (1.50 L) at 0-10° C. The nitrogen flow was set at 0.2 standard cubic feet per hour and agitation rate at 492-500 rpm. The addition took 1 hr 45 min. A vacuum was applied at 50-300 torr for a minimum of 3 hr to remove residual HCl from the system. Residual HCl remaining was <50 ppm (measured by ion chromatography). The agitation rate was set at 500 rpm and ≧90% nitric acid (0.079 kg, 1.2 eq) was added over 100 mins via a dip tube situated 13 cm above the surface of the reaction mixture. The reaction was mixed for 30 mins at 0-10° C. The reaction was followed by HPLC (starting material was undetected by HPLC after 30 mins). The cold reaction mixture was transferred over 1 hr to a mixture of IPA:heptane (13.7 L IPA, 1.65 L heptane) kept at 0-12° C. in a 20-L jacketed cylindrical reactor. The precipitated product was mixed at 0-10° C. overnight, filtered, washed with IPA:heptane (3.225 L IPA, 0.55 L heptane) followed by IPA (3.6 L). The product was dried at 40-42° C. to an LOD of ≦4.0% to provide 613 g (93% yield) of 9-nitrominocycline sulfate. The purity of the 9-nitrominocycline produced was 76.5%.

### Example of Reduction

### Preparation of 9-aminominocycline hydrochloride

In a 2-gallon stirred pressure reactor (Parr 455SS) equipped with double stage pitched blade style impellers (9.9 cm diameter), baffle and cooling coil, 5% palladium on charcoal 50% water wet (20 g) and 9-nitrominocycline sulfate (400 g) were charged. The pressure reactor was purged three times with nitrogen. Methanol (412 g) and purified water (2.1 kg) were charged using nitrogen pressure. The agitation rate was set to 345-355 rpm and the reaction mixture was cooled to 5-10° C. The chilled reaction mixture was hydrogenated under 70 psi of hydrogen for 10 hours. The completion of the reaction was monitored by HPLC (SM %<0.5%). The catalyst was filtered through 0.2 μm cartridge (Pall VFTR200-04M3S). The reactor, the lines and the filter were rinsed with cooled (0-10° C.) purified water (490 g). The clarified solution was transferred using vacuum into a 5-L jacketed cylindrical reactor equipped with a pitched blade style impeller (12.0 cm diameter), thermocouple and nitrogen inlet. The temperature of the solution was adjusted to 0-5° C. Sodium sulfite (0.24 g) was added followed by HCl reagent (267.8 g) added over 15 min. The pH was 0.9. The pH was adjusted to 3.8 to 4.2 using ammonium hydroxide 28% (254.4 g). During this pH adjustment, the temperature was kept below 10° C. The mixture was held for 2 hours at 0-10° C. The solids were filtered on a Büchner funnel (20 cm diameter) and washed with cooled purified water (245 g, adjusted to pH 3.8-4.2) and acetone (579 g). The product was dried under vacuum at 40-45° C. until LOD was ≦7%. The process provided 117 g in two crops (80 g+37 g, 43% overall yield from minocycline) of 9-aminominocycline HCl. The second crop material was recovered after allowing the mother liquors to stand overnight in the refrigerator. The purity of the 9-aminominocycline produced was 95.9% (first crop) and 96.3% (second crop).

### HPLC Analytical Methods

SMA % is starting material area percent; SMA is starting material area; P is product.

### Nitration

Calculation SMA %=SMA peak area×100%÷(SMA peak area+P peak area)

Sample preparation: Take 2-3 drops of the reaction mixture into a 2-mL HPLC sample vial and dilute with mobile phase A. Inject.

### LC Conditions

| Column: | Waters Symmetry Shield | | |
|---|---|---|---|
| | RP8 3.5μ (15 × 0.46) cm | | |
| Mobile phase: | A: 0.03 M KH₂PO₄ monobasic, | | |
| | pH 2 with H₃PO₄ | | |
| | B: 9:1 | | |
| | acetonitrile:water | | |
| Flow rate: | 0.8 mL/min | | |
| Detection wavelength: | 250 nm | | |
| Column oven temperature: | 35° C. | | |
| Sample volume injection: | 10 μL | | |
| | Time | Mobile | Mobile |
| Isocratic program: | (min) | Phase A (%) | Phase B (%) |
| | 0 | 90 | 10 |
| | 2 | 90 | 10 |
| | 30 | 45 | 55 |
| | 32 | 90 | 10 |
| | 38 | 90 | 10 |

Retention Times:

| | HPLC retention |
|---|---|
| Compound | time (min) |
| Minocycline hydrochloride | 3.90-4.30 |
| 9-Nitrominocycline | 15.10-15.50 |
| Impurity A | 17.40-17.80 |
| Impurity B | 9.65-10.05 |

### Hydrogenation

Calculation SMA %=SMA peak area×100%÷(SMA peak area+P peak area)

Sample preparation: Take 2-3 drops of the reaction mixture into a 2-mL HPLC sample vial and dilute with mobile phase A. Inject.

### LC Conditions

| Column: | Waters Symmetry Shield | | |
|---|---|---|---|
| | RP8 3.5μ (15 × 0.46) cm | | |
| Mobile phase: | A: 0.03 M KH₂PO₄ monobasic, | | |
| | pH 2 with H₃PO₄ | | |
| | B: 9:1 | | |
| | acetonitrile:water | | |
| Flow rate: | 0.8 mL/min | | |
| Detection wavelength: | 250 nm | | |
| Column oven temperature: | 35° C. | | |
| Sample volume injection: | 10 μL | | |
| | Time | Mobile | Mobile |
| Isocratic program: | (min) | Phase A (%) | Phase B (%) |
| | 0 | 96 | 4 |
| | 6 | 96 | 4 |
| | 20 | 50 | 50 |
| | 30 | 50 | 50 |
| | 33 | 96 | 4 |
| | 38 | 96 | 4 |

### Retention Times:

| | HPLC retention |
|---|---|
| Compound | time (min) |
| 9-Nitrominocycline | 17.40-17.80 |
| 9-Aminominocycline | 4.80-5.20 |

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

What is claimed is:

1. A method of preparing the compound of formula 1 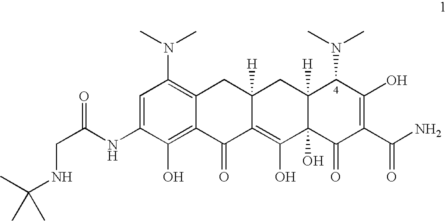
or a pharmaceutically acceptable salt thereof,
comprising:
(a) reacting nitric acid with the compound of formula 2, 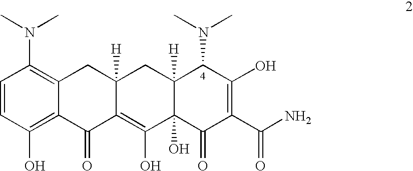
or a salt thereof, to produce a reaction mixture comprising an intermediate; and
(b) further reacting the intermediate to form the compound of formula 1, wherein the intermediate is isolated from the reaction mixture,
the method further comprising sparging with an inert gas prior to step (a).

2. The method of claim 1, wherein the inert gas is nitrogen.

3. The method of claim 2, wherein compound 2 is in contact with a reaction medium and compound 2 and the reaction medium are within a reactor vessel, wherein the reaction medium forms a surface defining a headspace portion above the surface and a subsurface portion beneath the surface, the method comprising (a) sparging the headspace portion without sparging the subsurface portion, (b) sparging the subsurface portion without sparging the headspace portion, or (c) sparging the headspace portion and the subsurface portion.

4. The method of claim 3, wherein compound 2 is dissolved in the reaction medium.

5. The method of claim 3, wherein the process comprises sparging the headspace portion without sparging the subsurface portion.

6. The method of claim 3, wherein the process comprises sparging the subsurface portion without sparging the headspace portion.

7. The method of claim 3, wherein the process comprises sparging the headspace portion and the subsurface portion.

8. The method of claim 3, wherein the salt of the compound of formula 2 is a hydrochloride, wherein sparging with nitrogen decreases the amount of hydrogen chloride in the reactor vessel.

9. The method of claim 3, wherein the amount of hydrogen chloride is decreased by up to 95%

10. The method according to claim 1 wherein the nitric acid has a concentration of at least 90%.

11. The method according to claim 1, wherein the nitric acid is present in a molar excess relative to the compound of formula 2 is at least 1.05 equivalents

12. The method according to claim 11, wherein the molar excess is 1.2 to 1.5 equivalents.

13. The method according to claim 1, wherein the reacting in (a) is in the presence of an acid.

14. The method according to claim 13, wherein the acid is sulfuric acid.

15. The method according to claim 1, wherein the reacting in (a) is at a temperature ranging from 0 to 15° C.

16. The method according to claim 1, wherein the at least one compound of formula 2 is chosen from a salt.

17. The method according to claim 16, wherein the salt of the at least one compound of formula 2 is chosen from hydrochloride, hydrobromide, hydroiodide, phosphoric, nitric, sulfuric, acetic, benzoic, citric, cystein, fumaric, glycolic, maleic, succinic, tartaric, sulfate, and chlorobenzensulfonate salts.

18. The method according to claim 16, wherein the salt of the at least one compound of formula 2 is chosen from sulfuric acid or HCl salts.

19. The method according to claim 1, wherein the nitration reaction is performed under vacuum of 50 to 300 torr at 3 to 7° C.

20. The method according to claim 18, wherein the intermediate is a sulfate salt.

21. The method according to claim 18 wherein the intermediate is the HCl salt.

22. The method according to claim 1, wherein the intermediate is the compound of formula 3, 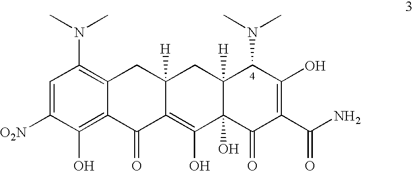
or a salt thereof.

23. The method according to claim 22, wherein the compound of formula 3 is present in an amount of at least 80% relative to the total amount of organic components, as determined by high performance liquid chromatography.

24. The method according to claim 22, wherein the reaction mixture includes the C₄-epimer of formula 3 in an amount less than 3% as determined by high performance liquid chromatography.

25. The method according to claim 22 wherein the reaction mixture includes minocycline in a range of less than 5% to 0.1%.

26. The method according to claim 25, wherein the reducing in (b) forms the compound of formula 4, 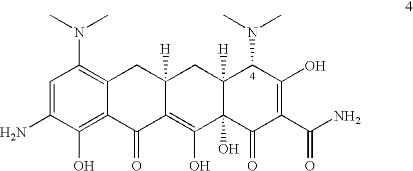
or a salt thereof.

27. The method according to claim 26, further comprising acylating the reduced intermediate.

28. The method of claim 1, wherein the reacting in (a) comprises providing the compound of formula 2 in an amount of at least 1 gram.

29. The method of claim 2, further comprising:
adjusting the temperature of the reaction mixture to 0-40° C.;
adding 5 to 20% of an antisolvent over 20 to 120 minutes to the reaction mixture,
wherein the antisolvent is added from or through a container fitted with a jacket,
wherein the the jacket temperature is adjusted to 0-40° C.;
adding the remainder of the antisolvent over 2-5 hrs to the reaction mixture while maintaining the reaction mixture temperature in the range of 0-40° C.;
stirring the reaction mixture for 1 hr-24 hours at 0-40° C.;
cooling the reaction mixture to 0-40 C, wherein the temperature to which the reaction mixture is cooled is lower than the temperature at which the reaction mixture is stirred; and
filtering the reaction mixture.

30. A method of preparing the compound of formula 1, tigecycline 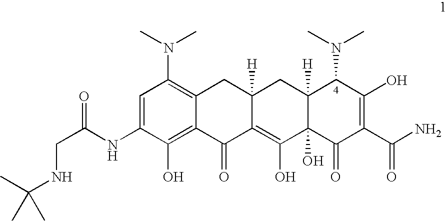
or a pharmaceutically acceptable salt thereof,
comprising:
(a) reacting the nitrating agent, nitric acid with the compound of formula 2, 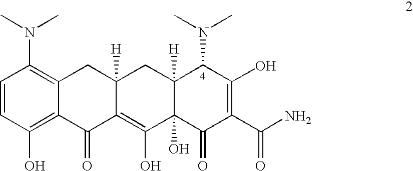
or a salt thereof, to produce a reaction mixture comprising an intermediate; and
(b) further reacting the intermediate to form the compound of formula 1,
wherein the intermediate is isolated from the reaction mixture,
the method further comprising sparging with an inert gas prior to step (a).

31. A method of preparing the compound of formula 1, tigecycline 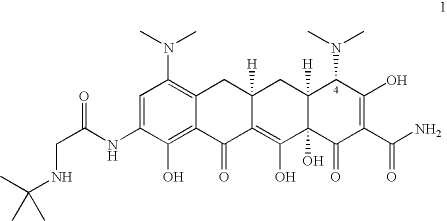
or a pharmaceutically acceptable salt thereof,
comprising:
(a) reacting the nitrating agent, nitric acid with the compound of formula 2, 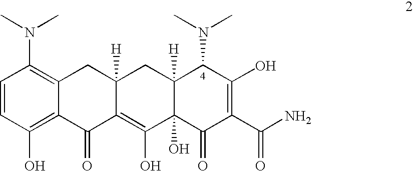
or a salt thereof, to produce a slurry; and
(b) further reacting the slurry to form the compound of formula 1,
the method further comprising sparging with an inert gas prior to step (a).

32. A method of preparing the compound of formula 3 or a salt thereof, 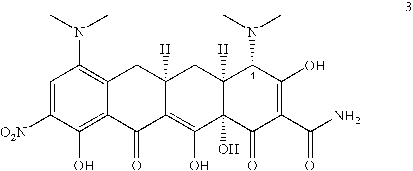
comprising:
reacting the nitrating agent, nitric acid with the compound of formula 2 or a salt thereof, 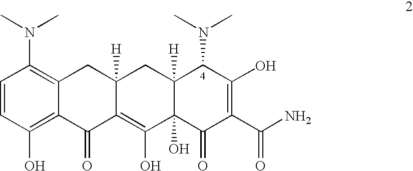
wherein the reacting is performed at a temperature ranging from 0 to 15° C.,
the method further comprising sparging with an inert gas prior to reacting the nitrating agent, nitric acid with the compound of formula 2 or a salt thereof.

33. A method of preparing the compound of formula 1, tigecycline 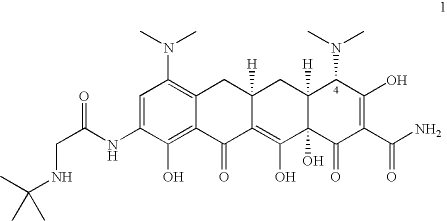
or a pharmaceutically acceptable salt thereof,
comprising:
(a) reacting a nitrating agent, nitric acid with the compound of formula 2 or a salt thereof at a temperature range of 3 to 7° C. to produce a reaction mixture comprising an intermediate and isolating said intermediate; and 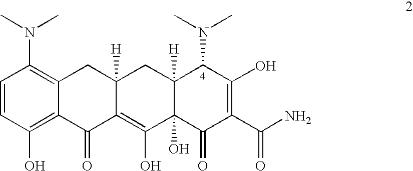
(b) reducing the intermediate in the presence of a Group VIII metal containing catalyst in aqueous methanol to form the at least one compound of formula 4 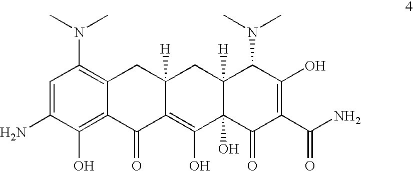
the method further comprising sparging with an inert gas prior to reacting the nitrating agent, nitric acid with the compound of formula 2 or a salt thereof.

34. The method according to claim 33, wherein the aqueous methanol is 80:20 water:methanol optionally in the presence of sulfuric acid.

35. The method according to claim 33, wherein the aqueous methanol is 99:1 methanol/water optionally in the presence of sulfuric acid.

36. The method according to claim 33 wherein the epi content of the intermediate is 1.42 to 1.96%.

37. The method according to claim 33 wherein the epi content of the compound of formula 4 is 2.45 to 2.95%.

38. A method for the preparation of 9-nitrominocycline comprising the steps of:
a. dissolving minocycline in sulfuric acid at (0 to 10° C.) over 1.5 to 2.0 hr under vacuum (50 to 300 torr) for a minimum of 3 hr;
b. adding nitric acid 90-100%(1.05-1.5 equivalents) above the surface at 3 to 7° C. over 100 to 180 minutes with stirring at 492-500 rpm with a holding time of 30 to 60 minutes to form a reaction mixture;
c. adding the reaction mixture to an 8.3:1 mixture of IPA:heptanes (vol:vol) over 1 hr), temperature of IPA:heptane mixture ( 0 to 12° C.);
d. isolating the product with a holding time during the product isolation of 2 hr to 24 hr; and optionally
e. drying the product isolated to less than or equal to 4% loss on drying at 40 to 45° C.,
the method further comprising sparging with an inert gas prior to step b to obtain a level of chloride of less than 150 ppm.

39. A method for the preparation of 9-aminominocycline comprising the steps:
f. forming a mixture of 9-nitrominocycline in 6 to 6.5 volumes of 80:20 water:methanol:
g. adding 5.0 to 10% palladium on carbon (50% water wet);
h. agitating at 345 to 355 rpm at 5 to 10° C. for 7 to 10 hr under 70 to 87 psi of hydrogen;
i. isolating the product having less than 0.5% of 9-nitrominocycline after holding for 2 to 24 hr, at a pH during product isolation of 3.8 to 4.2 at a temperature of less than 10° C. before isolating by filtration; and
j. drying the product to less than or equal to 7.0% loss on drying at 40 to 45° C.)
the method further comprising sparging with an inert gas prior to step a.
